(19) United States
(12) Patent Application Publication
(10) Pub. No.: US 2022/0251088 A1
(43) Pub. Date: Aug. 11, 2022

(54) Processes for Making PRMT5 Inhibitors

(71) Applicant: Prelude Therapeutics, Incorporated, Wilmington, DE (US)

(72) Inventor: Ganfeng Cao, Chadds Ford, PA (US)

(21) Appl. No.: 17/546,311

(22) Filed: Dec. 9, 2021

Related U.S. Application Data
(60) Provisional application No. 63/123,729, filed on Dec. 10, 2020.

(51) Int. Cl. C07D 487/04

(52) U.S. Cl. CPC C07D 487/04

(57) ABSTRACT

The disclosure provides processes for preparing the compound of formula (VIII) and pharmaceutically acceptable salts thereof. Intermediates useful in preparing the compound of formula (VIII) are also provided.

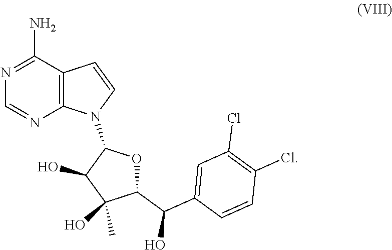 [SMILES: C[C@@]1(O)[C@@H]([C@H](O)c2ccc(Cl)c(Cl)c2)O[C@@H](n2ccc3c(N)ncnc32)[C@@H]1O]

### CROSS-REFERENCE TO RELATED APPLICATION

This application claims the benefit of U.S. Provisional Patent Application No. 63/123,729, filed on Dec. 10, 2020, the entirety of which is incorporated by reference herein.

### TECHNICAL FIELD

The disclosure is directed to methods of making PRMT5 inhibitors.

### BACKGROUND

Protein arginine methylation is a common post-translational modification that regulates numerous cellular processes, including gene transcription, mRNA splicing, DNA repair, protein cellular localization, cell fate determination, and signaling. Three types of methyl-arginine species exist: ω NG monomethylarginine (MMA), ω NG, NG asymmetric dimethylarginine (ADMA) and ω NG, N′G symmetric dimethylarginine (SDMA). The formation of methylated arginines is catalyzed by the protein arginine methyl transferases (PRMTs) family of methyltransferases. Currently, there are nine PRMTs annotated in the human genome. The majority of these enzymes are Type I enzymes (PRMT1, -2, -3, -4, -6, -8) that are capable of mono- and asymmetric dimethylation of arginine, with S-adenosylmethionine (SAM) as the methyl donor. PRMT-5, -7 and -9 are considered to be Type II enzymes that catalyze symmetric dimethylation of arginines. Each PRMT species harbors the characteristic motifs of seven beta strand methyltransferases (Katz et al., 2003), as well as additional “double E” and “THW” sequence motifs particular to the PRMT subfamily.

PRMT5 is as a general transcriptional repressor that functions with numerous transcription factors and repressor complexes, including BRG1 and hBRM, Blimp1, and Snail. This enzyme, once recruited to a promoter, symmetrically dimethylates H3R8 and H4R3. Importantly, the H4R3 site is a major target for PRMT1 methylation (ADMA) and is generally regarded as a transcriptional activating mark. Thus, both H4R3me2s (repressive; me2s indicates SDMA modification) and H4R3me2a (active; me2a indicates ADMA modification) marks are produced in vivo. The specificity of PRMT5 for H3R8 and H4R3 can be altered by its interaction with COPR5 and this could perhaps play an important role in determining PRMT5 corepressor status.

### Role of PRMTs in Cancer

Aberrant expression of PRMTs has been identified in human cancers, and PRMTs are considered to be therapeutic targets. Global analysis of histone modifications in prostate cancer has shown that the dimethylation of histone H4R3 is positively correlated with increasing grade, and these changes are predictive of clinical outcome.

PRMT5 levels have been shown to be elevated in a panel of lymphoid cancer cell lines as well as mantle cell lymphoma clinical samples. PRMT5 interacts with a number of substrates that are involved in a variety of cellular processes, including RNA processing, signal transduction, and transcriptional regulation. PRMT5 can directly modify histone H3 and H4, resulting in the repression of gene expression. PRMT5 overexpression can stimulate cell growth and induce transformation by directly repressing tumor suppressor genes. Pal et al., Mol. Cell. Biol. 2003, 7475; Pal et al. Mol. Cell. Biol. 2004, 9630; Wang et al. Mol. Cell. Biol. 2008, 6262; Chung et al. J Biol Chem 2013, 5534. In addition to its well-documented oncogenic functions in transcription and translation, the transcription factor MYC also safeguards proper pre-messenger-RNA splicing as an essential step in lymphomagenesis. Koh et al. Nature 2015, 523 7558; Hsu et al. Nature 2015 525, 384.

The discovery of cancer dependencies has the potential to inform therapeutic strategies and to identify putative drug targets. Integrating data from comprehensive genomic profiling of cancer cell lines and from functional characterization of cancer cell dependencies, it has been recently discovered that loss of the enzyme methylthioadenosine phosphorylase (MTAP) confers a selective dependence on protein arginine methyltransferase 5 (PRMT5) and its binding partner WDR77. MTAP is frequently lost due to its proximity to the commonly deleted tumor suppressor gene, CDKN2A. Cells harboring MTAP deletions possess increased intracellular concentrations of methylthioadenosine (MTA, the metabolite cleaved by MTAP). Furthermore, MTA specifically inhibits PRMT5 enzymatic activity. Administration of either MTA or a small-molecule PRMT5 inhibitor shows a preferential impairment of cell viability for MTAP-null cancer cell lines compared to isogenic MTAP-expressing counterparts. Together, these findings reveal PRMT5 as a potential vulnerability across multiple cancer lineages augmented by a common “passenger” genomic alteration.

### Role of PRMT5 in Hemoglobinopathies

The developmental switch in human globin gene subtype from fetal to adult that begins at birth heralds the onset of the hemoglobinopathies, b-thalassemia and sickle cell disease (SCD). The observation that increased adult globin gene expression (in the setting of hereditary persistence of fetal hemoglobin [HPFH] mutations) significantly ameliorates the clinical severity of thalassemia and SCD has prompted the search for therapeutic strategies to reverse gamma-globin gene silencing. Central to silencing of the gamma-genes is DNA methylation, which marks critical CpG dinucleotides flanking the gene transcriptional start site in adult bone marrow erythroid cells. It has been shown that these marks are established as a consequence of recruitment of the DNA methyltransferase, DNMT3A to the gamma-promoter by the protein arginine methyltransferase PRMT5. Zhao et al. Nat Struct Mol Biol. 2009 16, 304. PRMT5-mediated methylation of histone H4R3 recruits DNMT3A, coupling histone and DNA methylation in gene silencing.

PRMT5 induces the repressive histone mark, H4R3me2s, which serves as a template for direct binding of DNMT3A, and subsequent DNA methylation. Loss of PRMT5 binding or its enzymatic activity leads to demethylation of the CpG dinucleotides and gene activation. In addition to the H4R3me2s mark and DNA methylation, PRMT5 binding to the gamma-promoter, and its enzymatic activity are essential for assembly of a multiprotein complex on the gamma-promoter, which induces a range of coordinated repressive epigenetic marks. Disruption of this complex leads to reactivation of gamma gene expression. These studies provide the basis for developing PRMT5 inhibitors as targeted therapies for thalassemia and SCD.

The compound of formula (VIII), (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol, is a PRMT5 inhibitor that is described in U.S. Pat. No. 10,570,140.

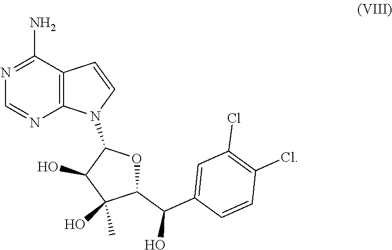 [SMILES: C[C@@]1(O)[C@@H]([C@H](O)c2ccc(Cl)c(Cl)c2)O[C@@H](n2ccc3c(N)ncnc32)[C@@H]1O]

A need exists for processes capable of preparing VIII and pharmaceutically acceptable salts thereof in high yields and with high stereochemical purity.

### SUMMARY

The disclosure provides methods of preparing the compound of formula (VIII) and pharmaceutically acceptable salts thereof in high yields and with high stereochemical purity.

In some aspects, the disclosure is directed to processes for preparing a compound of formula (VIII)

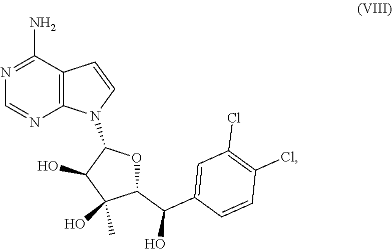 [SMILES: C[C@@]1(O)[C@@H]([C@H](O)c2ccc(Cl)c(Cl)c2)O[C@@H](n2ccc3c(N)ncnc32)[C@@H]1O]

or a pharmaceutically acceptable salt thereof, wherein the processes comprise any of the processes described herein.

In some aspects, the disclosure is directed to processes for preparing a compound of formula (III), comprising reacting a compound of formula (I) with a compound of formula (II) in the presence of an organic solvent:

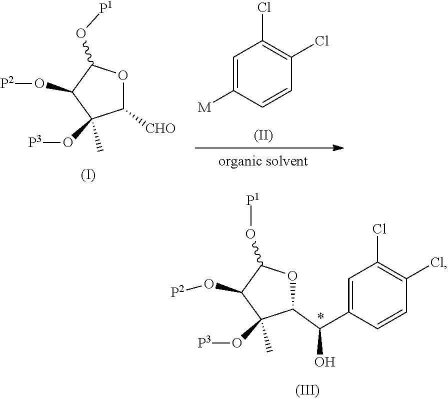

wherein M is a metal atom-containing moiety, a boronate ester, or a boronic acid; P¹ and P² are each, independently, a hydroxyl protecting group; or P¹ and P² together with the oxygen atoms to which they are attached form a 1,2-dihydroxyl protecting group; and P³ is H or a hydroxyl protecting group; or P² and P³ together with the oxygen atoms to which they are attached form a 1,2-dihydroxyl protecting group.

In other aspects, the processes of the disclosure further comprise treating the compound of formula (III) with a P⁴-Reagent System for a time and under conditions sufficient to provide a compound of formula (IV):

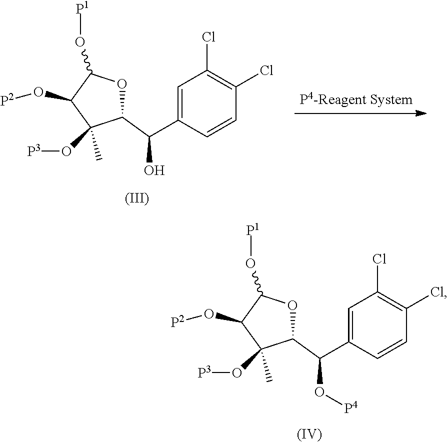

wherein the P⁴-Reagent System is a reagent that reacts with the compound of formula (III) to produce the compound of formula (IV), wherein P⁴ is an acid-stable, base-labile hydroxyl protecting group, and when P³ in formula (III) is H, P³ in formula (IV) is H or, together with P⁴, is an acid-stable, base-labile 1,3-dihydroxyl protecting group.

In other aspects, the processes of the disclosure further comprise reacting the compound of formula (IV) with aqueous acid to provide a compound of formula (V):

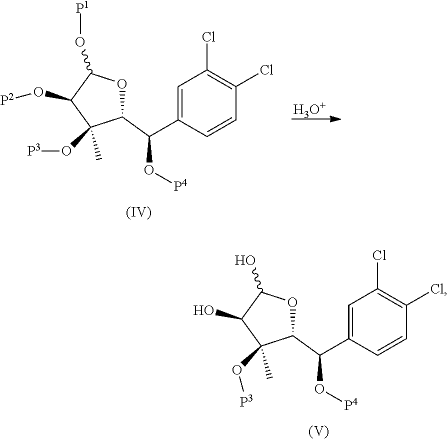

wherein P³ in the compound of formula (V) is H, or together with P⁴, forms an acid-stable, base-labile 1,3-dihydroxyl protecting group.

In other aspects, the processes of the disclosure further comprise reacting the compound of formula (V) with a compound of formula (VI), or a basic salt thereof, in the presence of a phosphine, an azodicarboxylate or derivative thereof, in the presence of an organic solvent, to produce a compound of formula (VII):

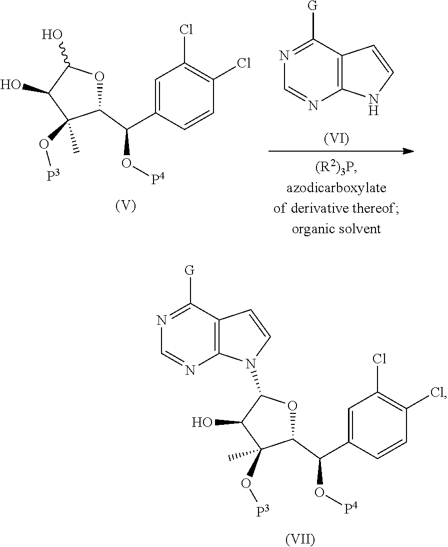

wherein G is a halogen or a masked amino group, each R² is independently C₁-C₆alkyl or aryl, and P³ is H, or together with P⁴, is an acid-stable, base-labile 1,3-dihydroxyl protecting group.

In some aspects, the disclosure is directed to processes for preparing a compound of formula (VIII) comprising reacting a compound of formula (VII) with ammonia for a time and under conditions sufficient to produce the compound of formula (VIII):

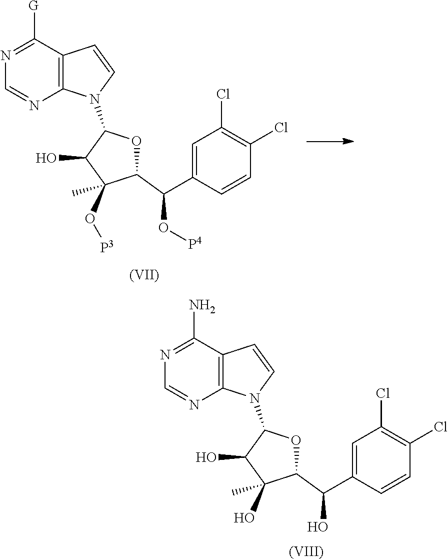

wherein G is halogen; P³ is H, and P⁴ is an acid stable, base-labile hydroxyl protecting group, or P³ and P⁴ together form an acid-stable, base-labile 1,3-dihydroxyl protecting group.

In other aspects, the disclosure is directed to processes for preparing a compound of formula (VIII) comprising reacting the compound of formula (VII) with a primary alkylamine for a time and under conditions sufficient to produce the compound of formula (VIII):

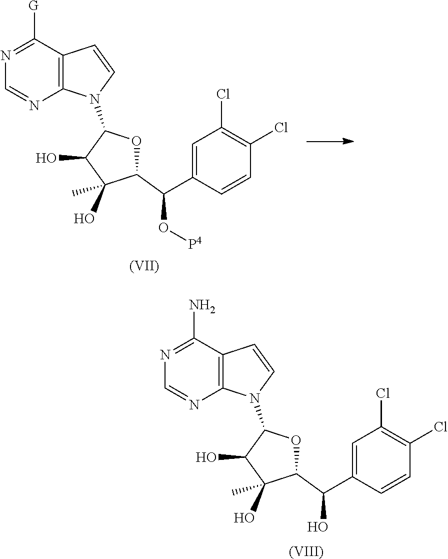

wherein G is a masked amino compound; P³ is H, and P⁴ is an acid stable, base labile hydroxyl protecting group, or P³ and P⁴ together form an acid-stable, base-labile 1,3-dihydroxyl protecting group.

### DETAILED DESCRIPTION OF ILLUSTRATIVE EMBODIMENTS

The disclosure may be more fully appreciated by reference to the following description, including the following definitions and examples. Certain features of the disclosed processes are described herein in the context of separate aspects, may also be provided in combination in a single aspect. Alternatively, various features of the disclosed processes that are, for brevity, described in the context of a single aspect, may also be provided separately or in any subcombination.

In the present disclosure the singular forms “a,” “an,” and “the” include the plural reference, and reference to a particular numerical value includes at least that particular value, unless the context clearly indicates otherwise. Thus, for example, reference to “an organic solvent,” “organic solvent,” “an appropriate organic solvent,” and the like is a reference to one organic solvent or a mixture of organic solvents. When a range of values is expressed, another embodiment includes from the one particular and/or to the other particular value. All ranges are inclusive and combinable.

The modifier “about” should be considered as disclosing the range defined by the absolute values of the two endpoints. For example, the expression “from about 2 to about 4” also discloses the range “from 2 to 4.” When used to modify a single number, the term “about” refers to plus or minus 10% of the indicated number and includes the indicated number. For example, “about 10° C.” indicates a range of 9° C. to 11° C., and “about 1” means from 0.9-1.1.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some aspects, the disclosure is directed to processes for preparing a compound of formula (III), comprising reacting a compound of formula (I) with a compound of formula (II) in the presence of an appropriate organic solvent:

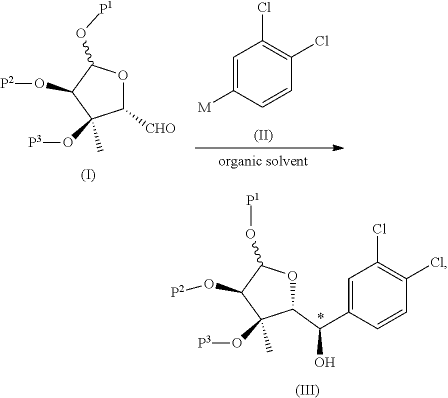

wherein M is a metal atom-containing moiety, a boronate ester, or a boronic acid; P¹ and P² are each, independently, a hydroxyl protecting group; or P¹ and P² together with the oxygen atoms to which they are attached form a 1,2-dihydroxyl protecting group; and P³ is H or a hydroxyl protecting group; or P² and P³ together with the oxygen atoms to which they are attached form a 1,2-dihydroxyl protecting group.

In the processes of the disclosure, the compound of formula (I) is an aldehyde that includes three groups P¹, P², and P³:

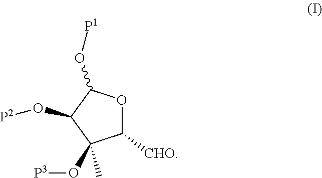

According to the present disclosure, P¹ and P² are each, independently, a hydroxyl protecting group; or P¹ and P² together with the oxygen atoms to which they are attached form a 1,2-dihydroxyl protecting group; and P³ is H or a hydroxyl protecting group; or P² and P³ together with the oxygen atoms to which they are attached form a 1,2-dihydroxyl protecting group.

As used herein, the term “hydroxyl protecting group” refers to a moiety that is bound to an oxygen atom of a compound (e.g., —O—P¹) such that the moiety (e.g., —P¹) can be removed under controlled conditions to yield a hydroxyl group (i.e., —OH). Hydroxyl protecting groups, methods of installing protecting groups, and methods for removing protecting groups are known to those of skill in the art and are described in, for example, Wuts, P. G. M., Greene's Protective Groups in Organic Synthesis, John Wiley & Sons, 5th ed. 2014.

In some embodiments, P¹ and P² are each, independently, a hydroxyl protecting group; or P¹ and P² together with the oxygen atoms to which they are attached form a 1,2-dihydroxyl protecting group.

In some embodiments, P¹ and P² are each, independently, a hydroxyl protecting group that is stable to (i.e., not removed during reaction) nucleophiles.

In some embodiments, P¹ and P² are each, independently, a hydroxyl protecting group that is stable during reaction with the compound of formula (II). Exemplary nucleophile-stable hydroxyl protecting groups include alkyl ethers, benzyl ethers, substituted benzyl ethers (e.g., p-methoxybenzyl ether), and silyl ethers (e.g., t-butyldimethylsilyl ether, trimethylsilyl ether).

In some embodiments P¹ or P² is an alkyl ether, such as, for example, a methyl ether, a methoxymethyl ether, a methylthiomethyl ether, a benzyloxymethyl ether, a substituted benzyloxymethyl ether, a t-butoxymethyl ether, a siloxymethyl ether, a methoxyethoxymethy ether, a tetrahydropyanyl ether, a 1-ethoxyethyl ether, a t-butyl ether, a trimethylsilyl ether, a t-butyldimethylsilyl ether, and the like.

In some embodiments P¹ or P² is a methyl ether. In some embodiments, P¹ is a methyl ether.

In some embodiments, P¹ and P² together with the oxygen atoms to which they are attached form a 1,2-dihydroxyl protecting group. In some embodiments, In some embodiments, P¹ and P² are each, independently, a hydroxyl protecting group that is stable to nucleophiles. In some embodiments, P¹ and P² together with the oxygen atoms to which they are attached form a 1,2-dihydroxyl protecting group that is stable during reaction with the compound of formula (II). Exemplary nucleophile-stable 1,2-dihydroxyl protecting groups include acetals (e.g., methylene acetal, ethylidene acetal, benzylidene acetal, p-methoxybenzylidene actetal, and the like), and ketals (e.g., acetonide and the like).

In some embodiments, P¹ and P² together with the oxygen atoms to which they are attached form an acetonide protecting group.

In some aspects, P³ is H or a hydroxyl protecting group; or P² and P³ together with the oxygen atoms to which they are attached form a 1,2-dihydroxyl protecting group.

In some embodiments, P³ is H.

In other embodiments, P³ is a hydroxyl protecting group.

In some embodiments, P³ is a nucleophile-stable hydroxyl protecting group.

In some embodiments, P³ is a methoxymethyl ether, a methylthiomethyl ether, a benzyloxymethyl ether, a substituted benzyloxymethyl ether, a t-butoxymethyl ether, a siloxymethyl ether, a methoxyethoxymethy ether, a tetrahydropyanyl ether, a 1-ethoxyethyl ether, a t-butyl ether, a trimethylsilyl ether, a tbutyldimethylsilyl ether and the like.

In some embodiments, P² and P³ together with the oxygen atoms to which they are attached form a 1,2-dihydroxyl protecting group.

In some embodiments, P² and P³ together with the oxygen atoms to which they are attached form a nucleophile-stable 1,2-dihydroxyl protecting group.

In some embodiments, P² and P³ together with the oxygen atoms to which they are attached form a an acetonide protecting group.

In some embodiments of the processes of the disclosure, the compound of formula (I) is a compound of formula (Ia):

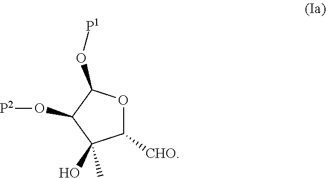

In some embodiments of the processes of the disclosure, the compound of formula (Ia) is a compound of formula (Ia-1):

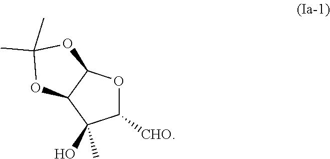

In some embodiments of the processes of the disclosure, the compound of formula (I) is a compound of formula (Ib):

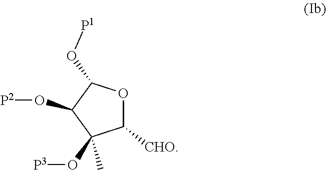

In some embodiments of the processes of the disclosure, the compound of formula (Ib) is a compound of formula (Ib-1):

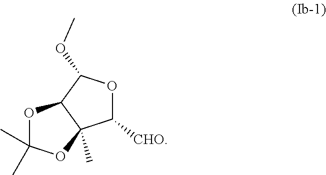

In the processes of the disclosure, M in the compound of formula (II) is a metal atom-containing moiety, a boronate ester, or a boronic acid.

In some embodiments, M is a metal atom-containing moiety. As used herein, the term “metal-atom-containing moiety” refers to a moiety that contains an alkali metal, an alkaline earth metal, a transition metal, a lanthanide, an actinide, aluminum, or a metaloid (e.g., B, Si). In some embodiments, metal-atom-containing moiety consists of only the metal atom or ion, such as, for example, Li⁺ or Na⁺. In other embodiments, the metal-atom-containing moiety consists the metal and other ligands, L.

In some embodiments, M is Li, MgL, ZnL, NiL₃, BL₂, CuL, SnL₃, Pd(L)₂, or Pd(L)₄, wherein L is a ligand. The ligand L may be any ligand that coordinates to the metal atom or metal ion and still renders the compound of formula (II) reactive with the aldehyde moiety of the compound of formula (I). Exemplary ligands, L, include halogens (e.g., —Cl, —Br, —I); alkoxides (e.g., —OCH₃); acetates (e.g., —OC(O)CH₃), phosphines (e.g., triphenylphosphine, tributylphosphine).

In some embodiments, M is —MgBr.

In some embodiments, M is a boronate ester. Exemplary boronate esters include —B(OCH₃)₂; —B(pinicol), and the like.

In some embodiments, M is a boronic acid, i.e., —B(OH)₂.

In some embodiments of the disclosed processes, the compound of formula (II) is a compound of formula (IIa):

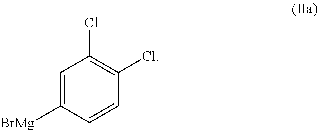

In some embodiments, the reaction of a compound of formula (I) with a compound of formula (II) is conducted in the presence of an appropriate organic solvent. In some embodiments, the organic solvent is an aprotic organic solvent. Exemplary aprotic organic solvents include Perfluorohexane, α,α,α-trifluorotoluene, pentane (Pent), hexane (Hex), cyclohexane (Cy), methylcyclohexane, decalin [c+t], dioxane, carbon tetrachloride, freon-11, benzene, toluene, triethyl amine, carbon disulfide, diisopropyl ether, diethyl ether (ether), t-butyl methyl ether (MTBE), chloroform, ethyl acetate, 1,2-dimethoxyethane (glyme), 2-methoxyethyl ether (diglyme), tetrahydrofuran (THF), methylene chloride, pyridine (Py), 2-butanone (MEK), acetone, hexamethylphosphoramide (HMPA), N-methylpyrrolidinone (NMP), nitromethane, dimethylformamide (DMF), acetonitrile, sulfolane, dimethyl sulfoxide (DMSO), propylene carbonate, and mixtures thereof.

In some embodiments, the aprotic organic solvent is diethyl ether, t-butyl methyl ether, or tetrahydrofuran, or mixtures thereof.

In some embodiments, the aprotic organic solvent is diethyl ether.

In other embodiments, the aprotic organic solvent is t-butyl methyl ether.

In other embodiments, the aprotic organic solvent is tetrahydrofuran.

In some embodiments of the processes of the disclosure, reacting a compound of formula (I) with a compound of formula (II) in the presence of an appropriate organic solvent is carried out in the presence of an additive. As used herein, the term “additive” refers to a compound or mixture of compounds that increases the yield, rate, or selectivity of the reaction.

In some embodiments, the additive is a Lewis acid. Exemplary Lewis acids include ZnCl₂, Sc(OTf)₃, TiCl₄, FeCl₃, CuCl₂, and the like.

In some embodiments, the Lewis acid is ZnCl₂.

In some embodiments of the processes of the disclosure, the reaction of a compound of formula (I) with a compound of formula (II) is conducted at a temperature in the range of about −25° C. to about 25° C. In some embodiments, the reaction is carried out at a temperature of about −10° C. to about 20° C.

In some aspects of the disclosed processes, reacting the compound of formula (I) with the compound of formula (II) produces the compound of formula (III):

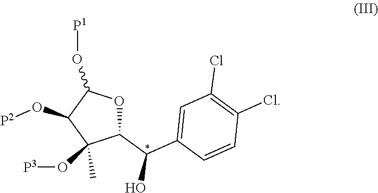

In some embodiments, the compound of formula (III) is a compound of formula (IIIa):

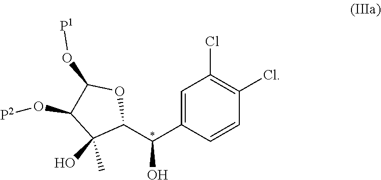

In other embodiments, the compound of formula (IIIa) is a compound of formula (IIIa-1):

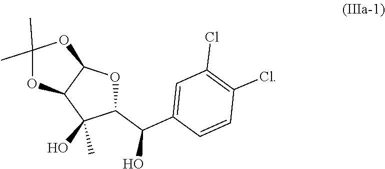 [SMILES: CC1(C)O[C@H]2O[C@H]([C@H](O)c3ccc(Cl)c(Cl)c3)[C@@](C)(O)[C@H]2O1]

In some embodiments of the disclosed processes, the compound of formula (III) is prepared by reacting a compound of formula (I) with a compound of formula (II), wherein M is a metal atom-containing moiety, a boronate ester, or a boronic acid, in the presence of an organic solvent for a time and under conditions sufficient to produce the compound of formula (III). In some embodiments, the compound of formula (I) is a compound of formula (Ia-1). In other embodiments, the compound of formula (I) is a compound of formula (Ib-1). In some embodiments, the compound of formula (II) is the compound of formula (IIa). In some embodiments, the conditions sufficient to produce the compound of formula (III) comprise conducting the reaction in the presence of a Lewis acid, such as, for example, ZnCl₂.

In some embodiments of the disclosed process, the compound of formula (III) has the formula (IIIa-1), the compound of formula (I) has the formula (Ia-1), the compound of formula (II) has the formula (IIa), the solvent is THF and the additive is ZnCl₂:

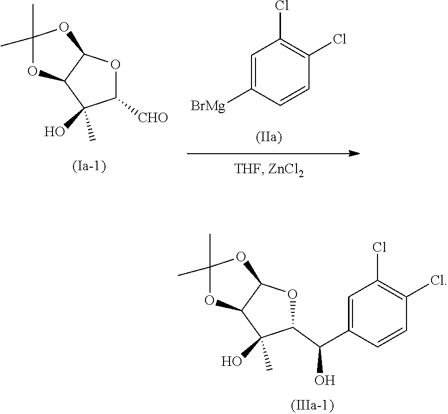

In other embodiments, the compound of formula (III) is a compound of formula (IIIb):

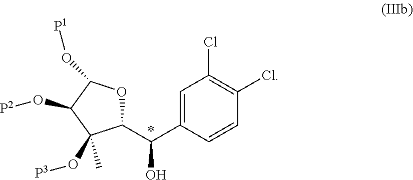

In other embodiments, the compound of formula (IIIb) is a compound of formula (IIIb-1):

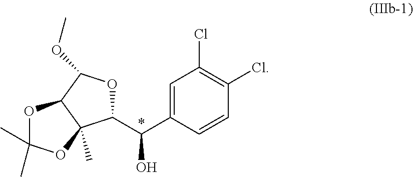 [SMILES: CO[C@@H]1O[C@H]([C@H](O)c2ccc(Cl)c(Cl)c2)[C@@]2(C)OC(C)(C)O[C@@H]12]

It will be clear to those of skill in the art that reacting the compound of formula (I) with the compound of formula (II) to give the compound of formula (III) results in the formation of an asymmetric benzylic carbon atom. This benzylic carbon atom is identified with an asterisk in some of the structures herein.

In some embodiments of the disclosed processes, reacting the compound of formula (I) with the compound of formula (II) gives the compound of formula (III) with an enantiomeric excess at the benzylic carbon atom (*) in the compound of formula (III) of at least 80%; at least 90%; at least 95%; at least 98%; at least 99%; at least 99.5%; at least 99.8%; or at least 99.9%. As used herein, the term “enantiomeric excess at the benzylic carbon” refers to the difference between the amount of one enantiomer at the benzylic carbon minus the amount of the other enantiomer at the benzylic carbon. For example, if a reaction produces 99% of enantiomer 1 and 1% of enantiomer 2, then the enantiomeric excess is 98%. Methods for determining the enantiomeric excess at the benzylic carbon atom will be known to those of skill in the art and include, for example, HPLC using a chiral stationary phase.

In some embodiments of the disclosed processes, reacting the compound of formula (I) with the compound of formula (II) gives the compound of formula (III) that is enriched in a particular diastereomer. In some embodiments, the diastereomeric excess in the compound of formula (III) is at least 80%; at least 90%; at least 95%; at least 98%; at least 99%; at least 99.5%; at least 99.8%; or at least 99.9%. As used herein, the term “diastereomeric excess” refers to the difference between the amount of one distereomer minus the amount of the other diastereomers. For example, if a reaction produces 99% of diaseteromer 1 and 1% total of other diastereomers, then the diastereomeric excess is 98%. Methods for determining the diastereomeric excess will be known to those of skill in the art and include, for example, HPLC using a chiral stationary phase.

In some aspects, the processes of the disclosure further comprise treating the compound of formula (III) with a P⁴-Reagent System for a time and under conditions sufficient to provide a compound of formula (IV):

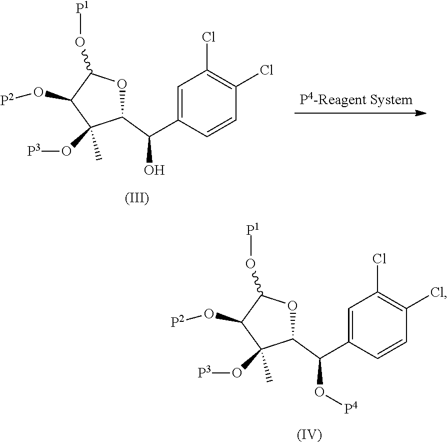

wherein the P⁴-Reagent System is a reagent that reacts with the compound of formula (III) to produce the compound of formula (IV), wherein P⁴ is an acid-stable, base-labile hydroxyl protecting group, and when P³ in formula (III) is H, P³ in formula (IV) is H or, together with P⁴, is an acid-stable, base-labile 1,3-dihydroxyl protecting group.

In some embodiments of the disclosed processes, the compound of formula (IV) is prepared by reacting a compound of formula (III) with a P⁴-Reagent System for a time and under conditions sufficient to provide a compound of formula (IV) wherein the P⁴-Reagent System is a reagent that reacts with the compound of formula (III) to produce the compound of formula (IV), wherein P⁴ is an acid-stable, base-labile hydroxyl protecting group. In some embodiments, the compound of formula (III) is a compound of formula (IIIa-1). In other embodiments, the compound of formula (III) is a compound of formula (IIIb-1).

As used herein, the term “P⁴-Reagent System” refers to a reagent, reagents, solvents, and/or other reaction conditions necessary to result in protection of the benzylic hydroxyl group of formula (III) with protecting group P⁴ to give formula (IV). As noted above, P⁴ is an acid-stable, base-labile hydroxyl protecting group.

In embodiments in which P³ in formula (III) is H, the “P⁴-Reagent System” is a reagent, reagents, solvents, and/or other reaction conditions necessary to result in protection of the benzylic hydroxyl group and the P³ hydroxyl group of formula (III) with protecting groups P³ and P⁴, wherein P³ and P⁴ together form an acid-stable, base-labile 1,3-dihydroxyl protecting group.

In some embodiments, the benzylic oxygen of formula (IV) and P⁴ form an ester, such as, for example, a benzoate ester, an acetate ester, or a pivaloate ester.

In some embodiments, the P⁴-Reagent System comprises a carboxylic acid chloride, an organic base, and an organic solvent.

In other embodiments, the P⁴-Reagent System comprises a carboxylic acid a carbodiimide or salt thereof, an additive, and an organic solvent.

In some embodiments, the carbodiimide is N,N′-Dicyclohexylcarbodiimide, N-Cyclohexyl-N′-(2-morpholinoethyl)carbodiimide, 1,3-Bis(trimethylsilyl)carbodiimide, N-Ethyl-N′-(3-dimethylaminopropyl)carbodiimide, Bis(4-methylphenyl)carbodiimide, N-Ethyl-N′-(3-dimethylaminopropyl)carbodiimide polymer-bound, N,N′-bis(2-methylphenyl)carbodiimide, N,N′-Diisopropylcarbodiimide, N,N′-Dicyclohexylcarbodiimide, polymer-bound, or a salt of any of the listed compounds.

In some embodiments, the additive is a pyridine or derivative thereof, such as N—N-dimethylaminopyridine (DMAP), or a benzotriazole derivative such as 1-hydroxybenzotriazole, or 1-hydroxy-7-azabenzotriazole.

In some embodiments, the organic solvent is dichloromethane, or tetrahydrofuran, ethyl acetate, isopropyl acetate, or acetone.

In some embodiments, P⁴ is

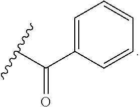 [SMILES: *C(=O)c1ccccc1]

In some embodiments, the P⁴-Reagent System is benzoic acid, a carbodiimide or salt thereof, an additive, and dichloromethane.

In some embodiments, the P⁴-Reagent System is benzoic acid, N-ethyl-N′-(3-dimethylaminopropyl)carbodiimide hydrochloride, dimethylaminopyridine, and dichloromethane.

In some embodiments of the disclosed processes, the compound of formula (III) is a compound of formula (IIIa) and the compound of formula (IV) is a compound of formula (IVa):

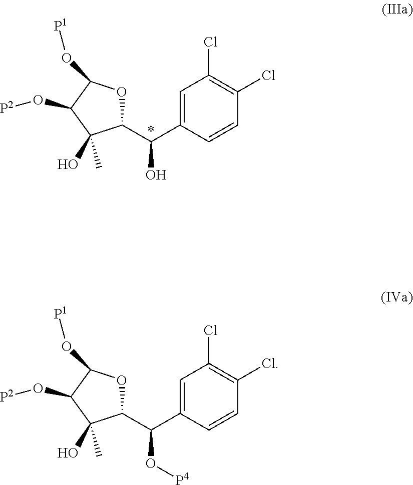

In some embodiments, the compound of formula (IVa) is the compound of formula (IVa-1):

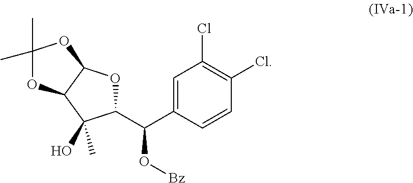

In some embodiments, the compound of formula (IIIa-1), is reacted with a P⁴ reagent system comprising benzoic acid, 1-ethyl-3-(3-dimethylaminopropyl)carbodiimide (EDCI) or a salt thereof, DMAP, and an appropriate organic solvent; to give the compound of formula (IVa-1):

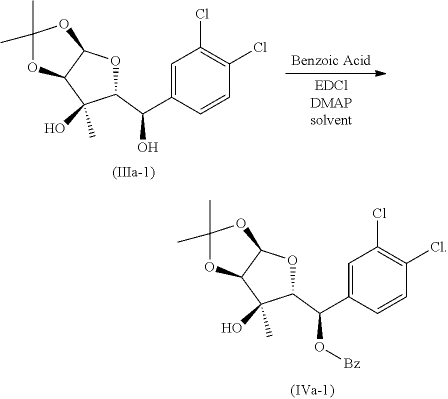

In some embodiments, the suitable organic solvent is dichloromethane.

In some embodiments of the disclosed processes, the compound of formula (III) is a compound of formula (IIIb) and the compound of formula (IV) is a compound of formula (IVb)

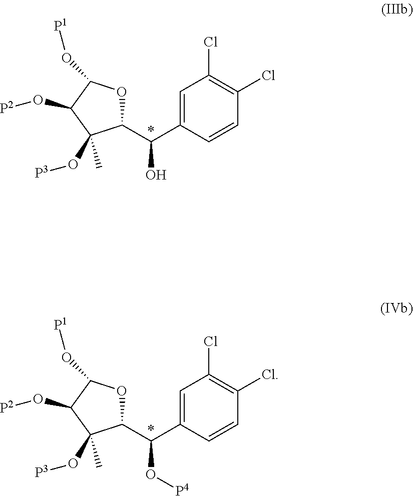

In some embodiments, the compound of formula (IVb) is the compound of formula (IVb-1):

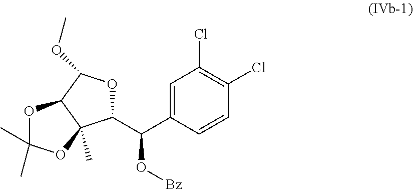

In some embodiments wherein P³ in formula (III) is H, reaction of the compound of formula (III) with the P⁴-Reagent System results in formation of a compound of formula (IV) wherein P³ and P⁴ together are an acid-stable, base-labile 1,3-dihydroxyl protecting group, such as, for example, a cyclic carbonate.

In some embodiments, the compound of formula (IV) is the compound of formula (IVc):

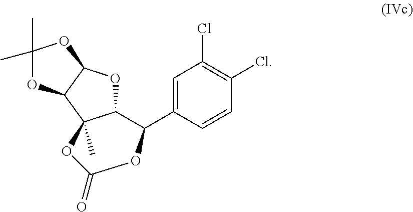

In some aspects, the processes of the disclosure further comprise treating the compound of formula (IV) with aqueous acid to provide a compound of formula (V):

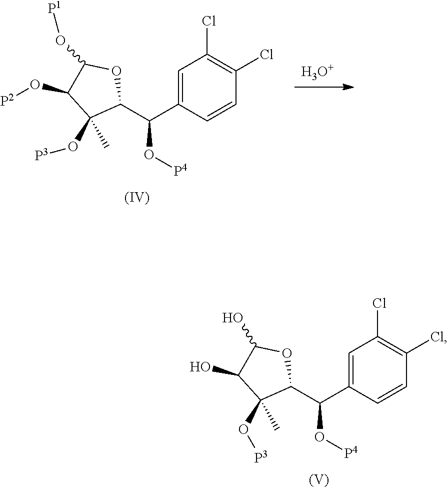

wherein P³ in the compound of formula (V) is H, or together with P⁴, forms an acid-stable, base-labile 1,3-dihydroxyl protecting group.

In some embodiments of the disclosed processes, the compound of formula (V) is prepared by treating the compound of formula (IV), (wherein P¹ and P² are each, independently, a hydroxyl protecting group; or P¹ and P² together with the oxygen atoms to which they are attached form a 1,2-dihydroxyl protecting group; and in formula (IV) P³ is H or a hydroxyl protecting group; or P² and P³ together with the oxygen atoms to which they are attached form a 1,2-dihydroxyl protecting group) with aqueous acid for a time and under conditions sufficient to produce the compound of formula (V) wherein P³ is H or P³ and P⁴ together form an acid-stable, base-labile 1,3-dihydroxyl protecting group. In some embodiments, the compound of formula (IV) has the formula (IVa-1). In some embodiments, the compound of formula (IV) has the formula (IVb-1).

In some embodiments wherein the compound of formula (IV) is the compound of formula (IVc), treatment with aqueous acid provides a compound of formula (Vc):

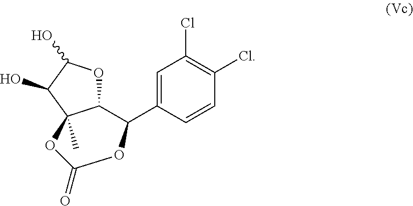

In some embodiments, the aqueous acid is a mixture of water and an acid that is capable of affecting the removal of acid-labile P¹, P², and, if present, an acid-labile P³ without affecting the removal of P⁴. Such acids will depend on the identities of P¹, P², and, if present, an acid-labile P³, and will be known to those skilled in the art as described in, for example, Wuts, P. G. M., Greene's Protective Groups in Organic Synthesis, John Wiley & Sons, 5th ed. 2014.

In some embodiments, the acid is a mineral acid. Non-limiting examples of mineral acids include HCl, H₃PO₄, and H₂SO₄.

In other embodiments, the acid is an acidic ion-exchange resin. Non-limiting examples of such resins include those sold under the tradenames Dowex (styrene divinylbenzene (gel) with sulfonic acid functional groups); Amberlite (Styrene-Divinylbenzene (DVB) gel or macroreticular, with sulfonic acid functional groups); and Amberlyst (styrene-divinylbenzene (macroreticular) with sulfonic acid functional groups).

In some embodiments, the aqueous acid is used in the presence of an organic solvent. Non-limiting examples of organic solvents that may be used in this regard include acetonitrile (ACN), THF, DMF, and alcohols such as methanol, ethanol and isopropanol.

In some embodiments, the aqueous acid is a mixture of H₂SO₄, water, and ACN.

In other embodiments, the aqueous acid is a mixture of water, acidic ion-exchange resin, and optionally an organic solvent.

In some embodiments, reaction formula (IV) with aqueous acid to provide a compound of formula (V) is conducted at a temperature between about 0° C. and about 100° C., preferably, between 45-55° C.

In some embodiments, the compound of formula (V) in the processes of the disclosure is the compound (Va):

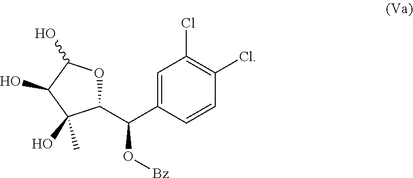

In some embodiments, the processes of the disclosure comprise reacting the compound of formula (IVa-1) with aqueous mineral acid in the presence of an organic solvent to give the compound of formula (Va):

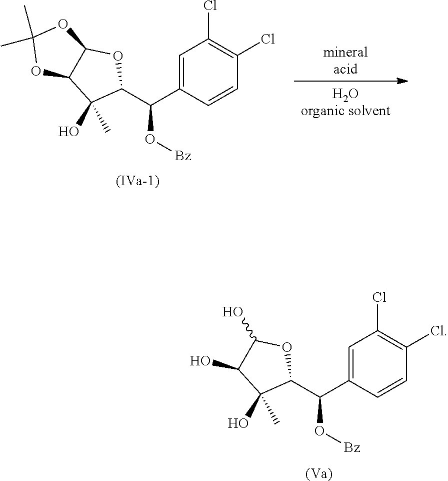

In some embodiments, the mineral acid is sulfuric acid (H₂SO₄) and the organic solvent is acetonitrile (ACN).

In some aspects, the processes of the disclosure further comprise reacting the compound of formula (V) with a compound of formula (VI), or a basic salt thereof, in the presence of a phosphine and an azodicarboxylate or derivative thereof, in the presence of an appropriate organic solvent, to produce a compound of formula (VII):

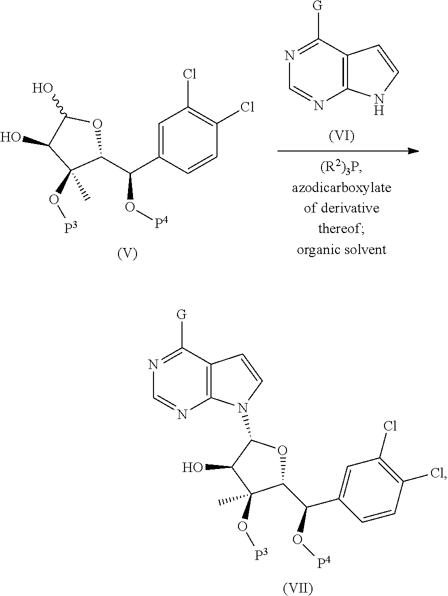

wherein G is a halogen or a masked amino group, each R² is independently C₁-C₆alkyl or aryl, and P³ is H, or together with P⁴, is an acid-stable, base-labile 1,3-dihydroxyl protecting group.

In some embodiments of the disclosed processes, the compound of formula (VII) is prepared by reacting the compound of formula (V) with a compound of formula (VI) or a salt thereof, in the presence of a phosphine (R²)₃P wherein each R² is independently C₁-C₆alkyl or aryl, an azodicarboxylate or derivative thereof, and an organic solvent, for a time and under conditions sufficient to produce the compound of formula (VII). In some embodiments, the compound of formula (V) is the compound of formula (Va).

In those embodiments wherein the compound of formula (V) is a compound of formula (Vc), reaction with a compound of formula (VI) under the conditions of the disclosure results in formation of a compound of formula (VIIc):

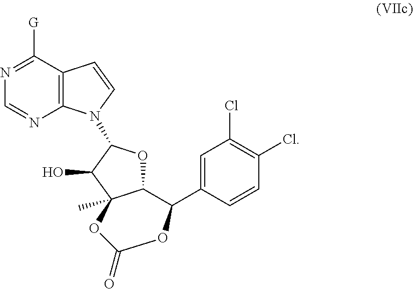

In some embodiments, an appropriate organic solvent is an aprotic organic solvent.

Gin the compound of formula (VI) or formula (VII) is a halogen or a masked amino group.

In some embodiments, G is a halogen, i.e., —Cl, —Br, or —I. In some embodiments, G is —Cl.

In embodiments wherein G is —Cl, the compound of formula (VI) is the compound of formula (VIa), or a basic salt thereof:

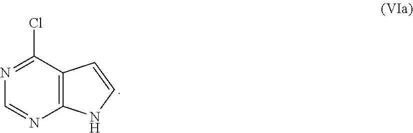

In some embodiments wherein G is —Cl, the compound of formula (VII) is the compound of formula (VIIa):

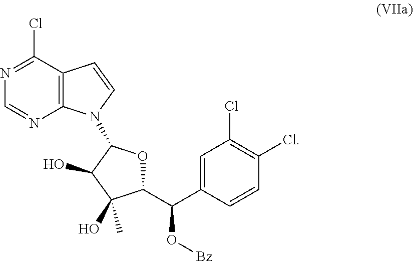

In other embodiments, Gin the compound of formula (VI) or formula (VII) is a masked amino group. As used herein, the term “masked amino group” refers to a group in which the atom of G that is attached to the pyrrolopyrimidine group in a nitrogen atom that is part of a larger group that that can be converted into a primary amino group, i.e., to convert G to —NH₂.

In some embodiments, Gin the compound of formula (VI) or formula (VII) is isoindol-2-yl-1,3-dionyl.

In embodiments wherein G is isoindol-2-yl-1,3-dionyl, the compound of formula (VI) is the compound of formula (VIb), or a basic salt thereof:

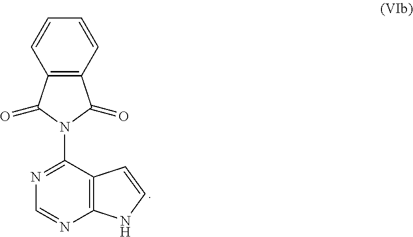 [SMILES: O=C1c2ccccc2C(=O)N1c1ncnc2[nH]ccc12]

In some embodiments wherein G is isoindol-2-yl-1,3-dionyl, the compound of formula (VII) is the compound of formula (VIIb):

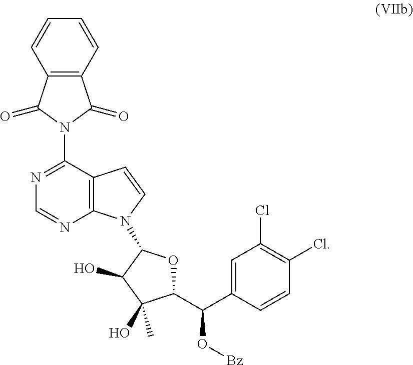

The phosphine used for reacting the compound of formula (V) with a compound of formula (VI) is any phosphine suitable for use in a Mitsunobu reaction. Such phosphines are known to those of skill in the art. In some embodiments, each R2 in the phosphine (R²)₃P used for reacting the compound of formula (V) with a compound of formula (VI) is independently C₁-C₆alkyl or aryl.

In some embodiments, the phosphine is (R²)₃P wherein R² is C₁-C₆alkyl, such as, for example, trimethylphosphien, triethylphosphine, tri-n-propylphosphine, tri-n-butylphosphine, and the like.

In some embodiments, the phosphine is tri-n-butylphosphine, (n-Bu)₃P.

In other embodiments, the phosphine is (R²)₃P wherein R² is aryl, such as, for example, triphenylphosphine, (p-dimethylaminophenyl)diphenylphosphine, diphenyl-2-pyridylphosphine, and the like.

The azodicarboxylate or derivative thereof used for reacting the compound of formula (V) with a compound of formula (VI) is any is azodicarboxylate or derivative thereof suitable for use in a Mitsunobu reaction. Such azodicarboxylates or derivatives thereof are known to those of skill in the art. In some embodiments, the azodicarboxylate or derivative thereof is diethylazodicarboxylate (DEAD), diisopropylazodicarboxylate (DIAD), or tetramethyl azodicarboxamide (TMAD).

In some embodiments, the azodicarboxylate or derivative thereof is diisopropylazodicarboxylate (DIAD).

In some embodiments, the azodicarboxylate or derivative thereof is tetramethyl azodicarboxamide (TMAD).

The organic solvent used for reacting the compound of formula (V) with a compound of formula (VI) is any organic solvent suitable for use in a Mitsunobu reaction. In some embodiments, the organic solvent is dichloromethane, chloroform, tetrahydrofuran, dioxane, diisoproplyether, DMF, acetonitrile, or a mixtures thereof. In some embodiments, the organic solvent is dichloromethane. In some embodiments, the organic solvent is tetrahydrofuran. In other embodiments, the organic solvent is a mixture of dichloromethane and tetrahydrofuran.

In some embodiments, the temperature used for reacting the compound of formula (V) with a compound of formula (VI) is between about 0° C. and 50° C. In some embodiments, the temperature is about 25° C. In other embodiments, the temperature is between about 0° C. to about 15° C.

In some embodiments, the processes of the disclosure comprise reacting the compound of formula (Va) with a salt of the compound of formula (Via—M wherein M is Na, Li, K, or Ca) (formed by reaction of the compound of formula (VIa) with strong base) in the presence of tetramethylazodicarboxarmide (TMAD) and tributylphosphine to give to the compound of formula (VIIa):

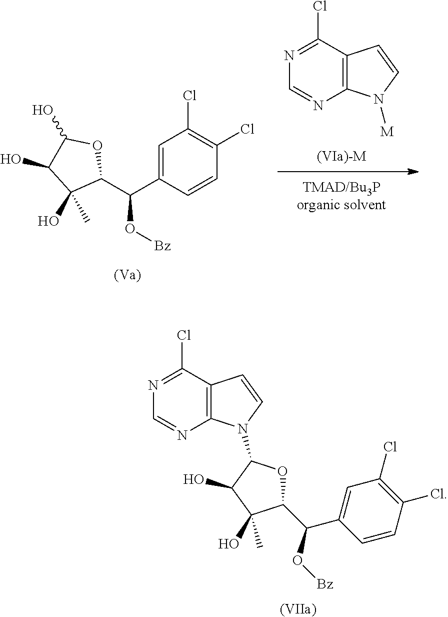

In some embodiments, the compound (VIa-M) is a sodium salt (i.e., M=Na).

In some embodiments, the strong base is NaH. In some embodiments, the organic solvent is acetonitrile.

In some embodiments, the reaction of a compound of formula (V) with a compound of formula (VI) proceeds through an epoxide intermediate having the formula IX. Where the compound of formula (V) is a compound of formula (Va), the epoxide intermediate has the formula (IXa):

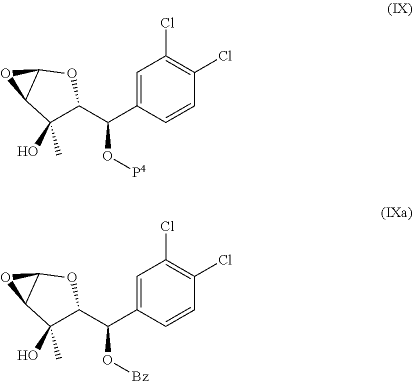

In some embodiments, the compound of formula (IX) or (IXa) is isolated prior to reaction with a compound of formula (VI).

In some aspects, the processes of the disclosure for preparing the compound of formula (V) further comprise converting the compound of formula (V) to an epoxide of formula (IX) by reacting the compound of formula (V) with a phosphine, an azodicarboxylate or derivative thereof, in the presence of an appropriate organic solvent:

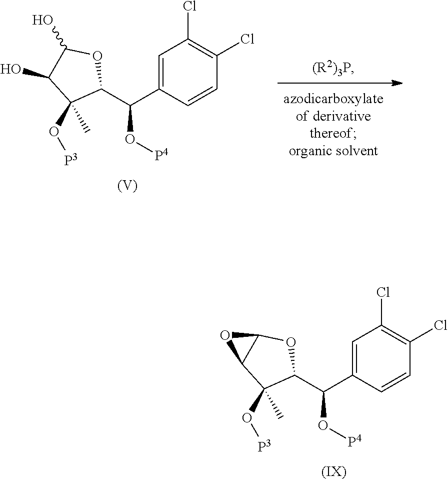

wherein each R² is independently C₁-C₆alkyl or aryl, and P³ is H, or wherein P³ and P⁴ together form an acid-stable, base-labile 1,3-dihydroxyl protecting group.

In some embodiments, the organic solvent is an aprotic organic solvent.

In some embodiments, the processes further comprise reacting the compound of formula (IX) with a compound of formula (VI), or a basic salt thereof, in an organic solvent to give a compound of formula (VII):

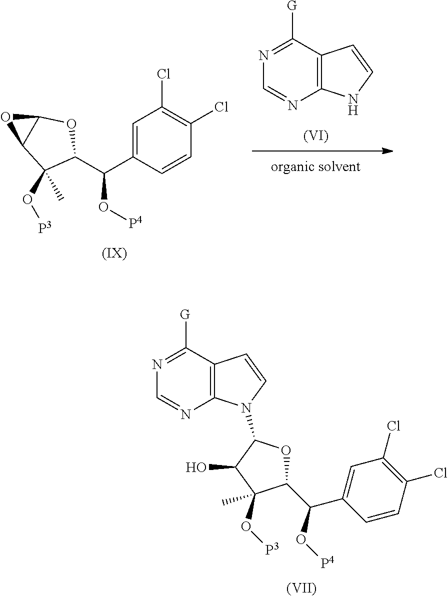

wherein G is a halogen or a masked amino group, and P³ is H, or wherein P³ and P⁴ together form an acid-stable, base-labile 1,3-dihydroxyl protecting group.

In some embodiments, the compound of formula (IX) is a compound of formula (IXa).

In some aspects, the processes of the disclosure further comprise converting the compound of formula (VII), wherein G is halogen and P³ is H, or wherein P³ and P⁴ together form an acid-stable, base-labile 1,3-dihydroxyl protecting group, to a compound of formula (VIII):

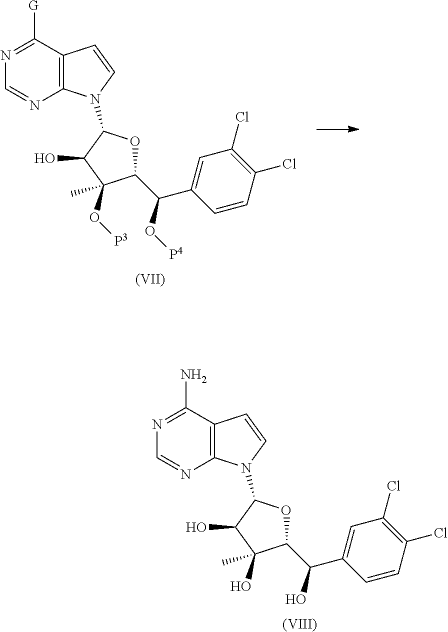

by reacting the compound of formula (VII) with ammonia.

In some embodiments, the disclosure is directed to processes for preparing a compound of formula (VIII) comprising reacting a compound of formula (VII) wherein G is halogen; P³ is H, and P⁴ is an acid stable, base-labile hydroxyl protecting group. with ammonia for a time and under conditions sufficient to produce the compound of formula (VIII). In some embodiments, the compound of formula (VII) is a compound of formula (VIIa).

In those embodiments wherein the compound of formula (VII) is a compound of formula (VIIc), reaction with ammonia under the conditions of the disclosure results in formation of a compound of formula (VIII).

In some embodiments wherein G is a halogen, G in formula (VII) is —Cl.

In some embodiments wherein G is a halogen, P⁴ in formula (VII) is -Bz (i.e. —OP⁴ is a benzoate ester).

In some embodiments wherein G is a halogen, the conversion of formula (VII) to formula (VIII) is accomplished by treating formula (VII) with aqueous ammonium hydroxide in an appropriate organic solvent at a suitable temperature and pressure. In some embodiments, the suitable organic solvent is a water miscible organic solvent that does not react with ammonium hydroxide. In some embodiments, the suitable organic solvent is 1,4-dioxane, or THF. In some embodiments, the reaction is conducted at a temperature between 25° C. and 125° C. In some embodiments, the reaction is carried out at elevated pressure, such as provided by conducting the reaction in a PARR apparatus.

In some embodiments, the processes of the disclosure comprise reacting the compound of formula (VIIa) with ammonia in an organic solvent to give the compound of formula (VIII):

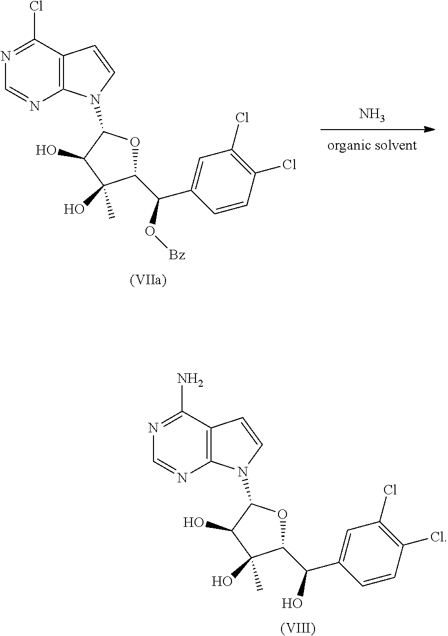

In some embodiments the ammonia is aqueous (i.e., NH₄OH). In other embodiments, the organic solvent is 1,4-dioxane. In some embodiments, the ammonia is aqueous (i.e., NH₄OH) and the organic solvent is 1,4-dioxane.

In some embodiments, the processes of the disclosure comprise reacting the compound of formula (Va) with the compound of formula (VIb) in the presence of diisopropylazodicarboxylate (DIAD), tributylphosphine, and an appropriate organic solvent to give the compound of formula (VIIb):

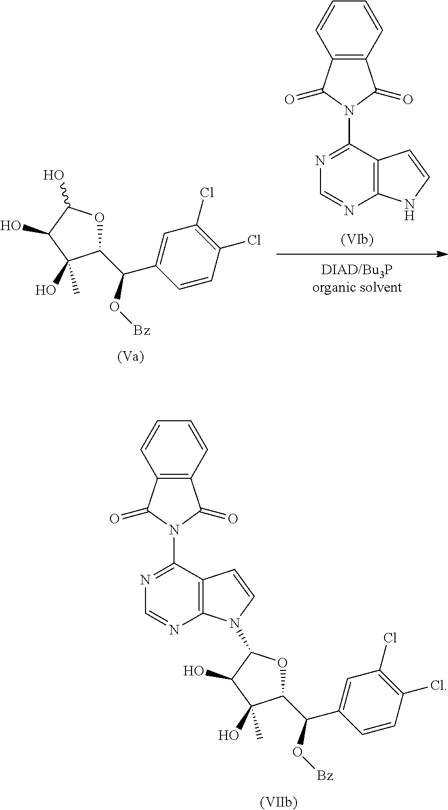

In some embodiments, the organic solvent is dichloromethane (DCM). In other embodiments, the solvent is THF. In yet other embodiments, the solvent is a mixture of THF and DCM.

In other aspects, the processes of the disclosure further comprise converting the compound of formula (VII), wherein G is a masked amino group and P³ is H, or wherein P³ and P⁴ together form an acid-stable, base-labile 1,3-dihydroxyl protecting group, to a compound of formula (VIII):

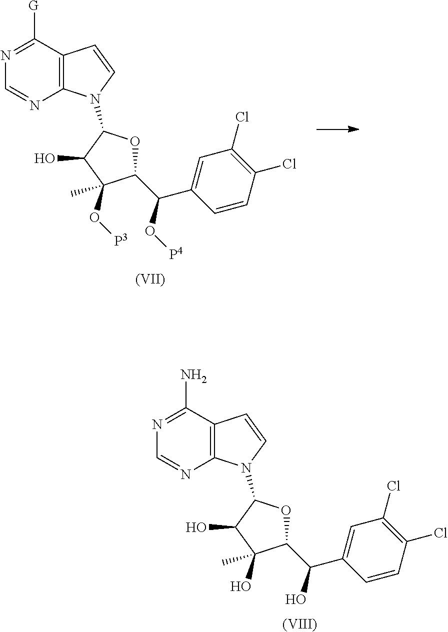

by reacting the compound of formula (VII) with a primary alkyl amine.

In some embodiments, the disclosure is directed to processes for preparing a compound of formula (VIII) comprising reacting the compound of formula (VII) wherein G is a masked amino compound; P³ is H, and P⁴ is an acid stable, base labile hydroxyl protecting group, with a primary alkylamine for a time and under conditions sufficient to produce the compound of formula (VIII) In some embodiments, the compound of formula (VII) is a compound of formula (VIIb).

In those embodiments wherein the compound of formula (VII) is a compound of formula (VIIc), reaction with ammonia under the conditions of the disclosure results in formation of a compound of formula (VIII).

In some embodiments wherein G is a masked amino group, G in formula (VII) is isoindol-2-yl-1,3-dionyl.

In some embodiments wherein G is a masked amino group, P⁴ in formula (VII) is -Bz (i.e. —OP⁴ is a benzoate ester).

In some embodiments wherein G is a masked amino group, the conversion of formula (VII) to formula (VIII) is accomplished by treating formula (VII) with a primary amine in an appropriate organic solvent at a suitable temperature. In some embodiments, the suitable organic solvent is an organic solvent that does not react with the primary amine. In some embodiments, the suitable organic solvent is methanol, ethanol, isopropanol, and the like. In some embodiments, the reaction is conducted at a temperature between 25° C. and 125° C., preferably about 65-75° C.

In some embodiments, the compound of formula (VIIb) is converted to the compound of formula (VIII) by reaction with n-butyl amine in an appropriate organic solvent:

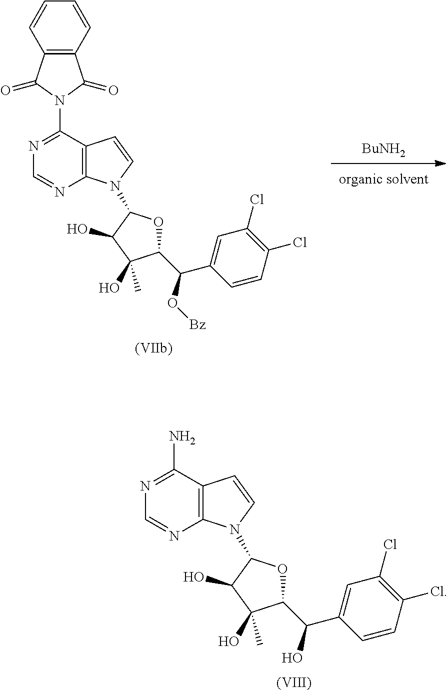

In some embodiments, the reaction of (VIIb) with n-butyl amine is carried out in methanol. In other embodiments, the reaction is carried out at about 65-75° C. In some embodiments, the reaction is carried out in methanol at about 65-75° C.

In some embodiments of the conversion of the compound of formula (VII) to a compound of formula (VIII), the compound of formula (VIII) is isolated by conversion to a solid salt (such as, for example the HCl salt) followed by filtration of the solid salt. Preparation of and isolation of various salts of the compound of formula (VIII) are described in WO2020168125, which is incorporated by reference herein. In some embodiments, the HCl salt of the compound or formula (VIII) is prepared by treating the crude reaction mixture with HCl in an appropriate solvent, such as, for example, by treating the crude reaction mixture with concentrated HCl in ethanol. In such embodiments, the HCl salt of the compound of formula (VIII) may be isolated by filtration from the reaction mixture.

Thus, in some embodiments, the conversion of formula (VIIa) to the HCl salt of formula VIII may be represented as:

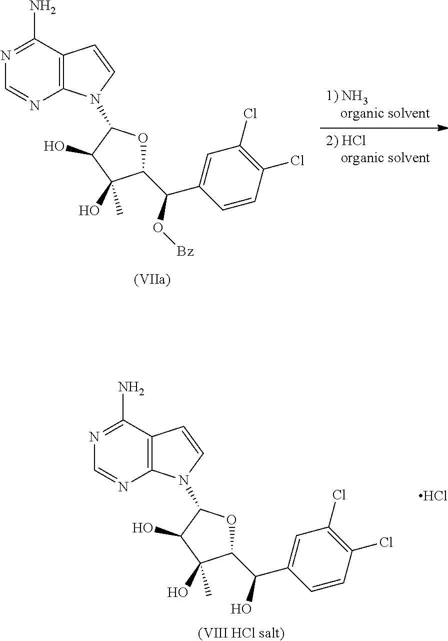

Similarly, in some embodiments, the conversion of formula (VIIb) to the HCl salt of formula (VIII) may be represented as:

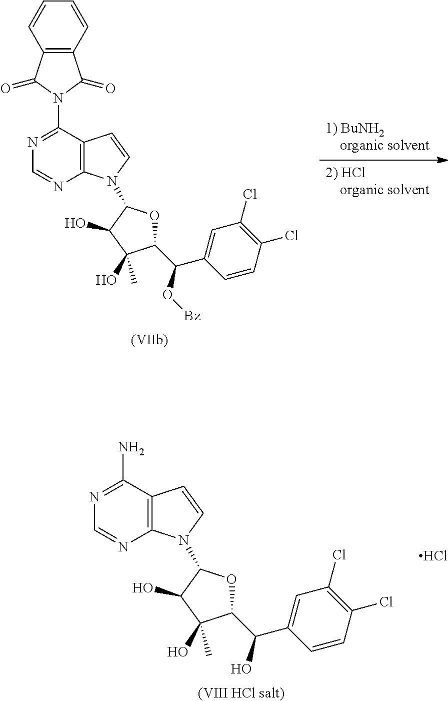

In embodiments of the disclosed processes in which the compound of formula (VIII) is isolated as a solid salt (such as, for example, a HCl salt, a phosphate salt, a sulfate salt, oxalate salt, oxalate salt, maleate salt), the salt may be converted to the formula (VIII) free base by reaction with a suitable base in the presence of an appropriate solvent. In some embodiments, the free base of formula (VIII) is obtained by treating the HCl salt of formula (VIII) with aqueous base, such as, for example, aqueous ammonium hydroxide:

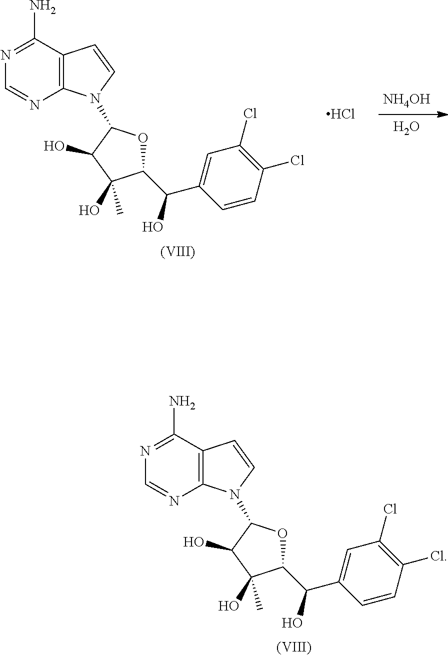

In some embodiments, this reaction is conducted at a temperature from about 10° C. to about 50° C., preferably from about 15° C. to about 35° C.

In some aspects, the processes of the disclosure further comprise reacting the compound of formula (VIII) with an acid to form a pharmaceutically acceptable salt of the compound of formula (VIII). In some embodiments, the pharmaceutically acceptable salt of the compound of formula (VIII) is a HCl salt, a phosphate salt, a sulfate salt, oxalate salt, oxalate salt, or maleate salt. The pharmaceutically acceptable salt may be prepared treating the compound of formula (VIII) with a suitable acid in the presence of suitable solvent.

In some embodiments, the processes of the disclosure further comprises reacting the compound of formula (VIII) with maleic acid in a solvent to produce the a pharmaceutically acceptable salt of the compound of formula (VIII) that is a compound of formula (VIIIa):

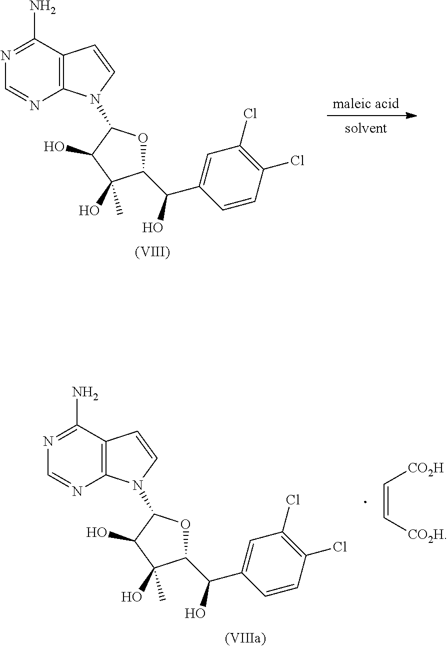

In some embodiments, the solvent used for the conversion of (VIII) to (VIIIa) is an alcohol, such as, for example methanol, ethanol, isopropanol, and the like. In some embodiments, the solvent is ethanol. In some embodiments, the conversion is conducted at a temperature of from about 0° C. to about 75° C., preferably between about 35-50° C.

In some embodiments, the disclosure is directed to a process for preparing a compound of formula (VIII) or a pharmaceutically acceptable salt thereof, wherein the process comprises any process disclosed herein.

In some aspects, the processes of the disclosure provide the compound of formula (VIII) or a pharmaceutically acceptable salt thereof, such as (VIIIa), in high stereoisomeric purity. That is, the compound of formula (VIII), or a pharmaceutically acceptable salt thereof, such as (VIIIa), is obtained predominantly as the stereoisomer having the absolute configuration shown below:

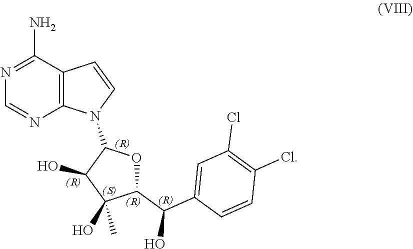

In some embodiments, the diastereomeric excess in the compound of formula (VIII) or a pharmaceutically acceptable salt thereof, such as (VIIIa), is at least 80%; at least 90%; at least 95%; at least 98%; at least 99%; at least 99.5%; at least 99.8%; or at least 99.9%. In some embodiments, the enantiomeric excess in the compound of formula (VIII) or a pharmaceutically acceptable salt thereof, such as (VIIIa), is at least 80%; at least 90%; at least 95%; at least 98%; at least 99%; at least 99.5%; at least 99.8%; or at least 99.9%. Methods of determining diastereomeric excess and enantiomeric excess are known to those skilled in the art, and include, for example, HPLC methods such as those described herein.

The following Examples are provided to illustrate aspects of the invention and are not intended to be limiting.

### EXAMPLES

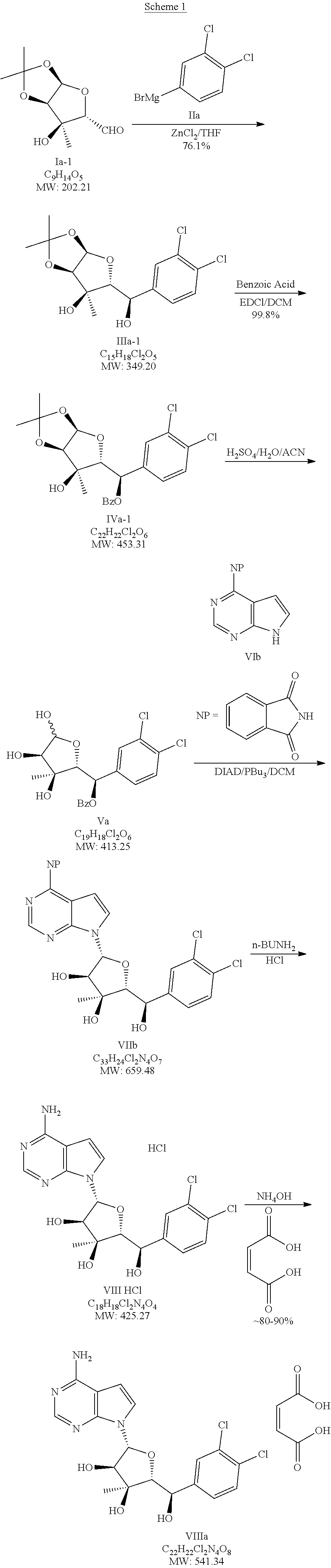

Summary: Zinc chloride mediated Grignard addition to aldehyde Ia-1 at 0° C. provided diol IIIa-1, with diastereoselectivity >99.7% to 0.3%. A simple slurry of the crude IIIa-1 can efficiently remove the undesired diastereomer (<0.10%). IIIa-1 is a white crystalline solid, and can be easily isolated, transferred and stored. Subsequent chemoselective protection of the benzylic alcohol of IIIa-1 with benzoic acid provided benzoate IVa-1 quantitively. IVa-1 was then hydrolyzed under acidic conditions to afford triol Va as a white powder. Crude Va was then submitted to a glycosylation reaction with N-protected pyrrolopyrimidine VIb via Mitsunobu reaction conditions to form nucleoside derivative VIIb. Without isolation or purification, the crude VIIb was treated with n-butyl amine to remove the N-protecting group, as well as the benzoyl group on the benzylic alcohol. Addition of hydrochloric acid afforded the VIII HCl salt as a crystalline solid. The yield from IIIa-1 to VIII HCl salt is ˜60%. VIII HCl salt was then neutralized to its freebase, followed by salt formation with maleic acid to give VIIIa (VIII maleate). The overall yield from Ia-1 to VIIIa is 36-41%.

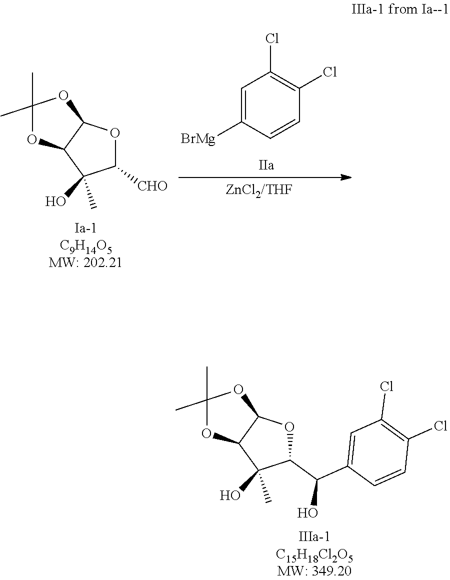

Preparation of Grignard reagent (IIa): To a 4-neck reactor charged with Mg turnings (350.7 g, 14.4 mol) under an N₂ atmosphere was added 10-20% of the total volume of a solution of 1-bromo-3,4-dichlorobenzene (2.26 kg, 10.0 mol) in THF (6.7 L). The mixture was cooled to 20° C. and a grain of I₂ was added [note: the temperature rose to 60° C. then cooled back down to 20-30° C.; initation was confirmed by observing a color change of the mixture from brown to gray]. The remainder of the solution of 1-bromo-3,4-dichlorobenzene in THF was added to the reaction mixture, dropwise, such that the temperature was maintained at ˜20° C. The mixture was stirred at this temperature for at least 2 h and used directly without further manipulation.

Grignard addition to aldehyde: To a solution of ZnCl₂ (1.0 M in THF, 3.7 L, 3.7 mol, 1.5 eq) cooled to −10-−5° C. under N₂ atmosphere was added (3,4-dichlorophenyl)magnesium bromide (1.5 M in THF, 7.9 L, 11.9 mol, 4.0 eq, prepared above) over 2 h such that the temperature was maintained below 0° C. The mixture was allowed to warm to 15° C. over 1 h then cooled to −10° C. Ia-1 (500.0 g, 2.47 mol, 1.0 eq) in THF (2.5 L) was added dropwise over 40 min to the cooled solution, with the temperature of the reaction maintained at <0° C. After addition was completed, the reaction mixture was warmed to 10° C. over 40 min then stirred at 15-20° C. for no less than 30 min. TLC indicated complete consumption of the starting material (60% EtOAc/Heptane). A solution of 20% aq. NH₄Cl (1.0 L) was slowly added to the mixture and the temperature of the contents was kept below 20° C. The reaction mixture was stirred for 20 min then filtered over celite. The resultant cake was washed with EtOAc (3.0 L) and the filtrate added back to the reactor. The organic phase was separated. The cake was washed with additional EtOAc (3.0 L) and the filtrate was used to extract the aqueous solution. The combined organic layers were washed with 10% aq. NaCl (2.5 L) then dried over anhydrous Na₂SO₄ (500 g). The drying agent was filtered and concentrated to give crude product which was then slurried with MTBE/Heptane (1:2, 5.0 L) at 20-25° C. for 1.5 h. The mixture was cooled to 5° C. and stirred for at least 30 min. The mixture was filtered, the cake washed with heptane (1.0 L), and the solid dried in vacuo at <45° C. to afford (3aR,5R,6R,6aR)-5-((R)-(3,4-dichlorophenyl)(hydroxy)methyl)-2,2,6-trimethyltetrahydrofuro[2,3-d][1,3]dioxol-6-ol (IIIa-1) (656.8 g, 1.87 mol, 76.1%).

¹H NMR (400 MHz, CDCl₃-d3): δ 7.53 (d, J=4.0 Hz, 1H), 7.41 (d, J=8.0 Hz, 1H), 5.25 (dd, J=4.0, 8.0 Hz, 1H), 5.71 (d, J=4.0 Hz, 1H), 4.68 (d, J=8.0 Hz, 1H), 4.18 (d, J=4.0 Hz, 1H), 3.65 (d, J=8.0 Hz, 1H), 2.97 (s, 1H), 2.90 (d, J=4.0 Hz, 1H), 1.52 (s, 3H), 1.42 (s, 3H), 1.33 (s, 3H).

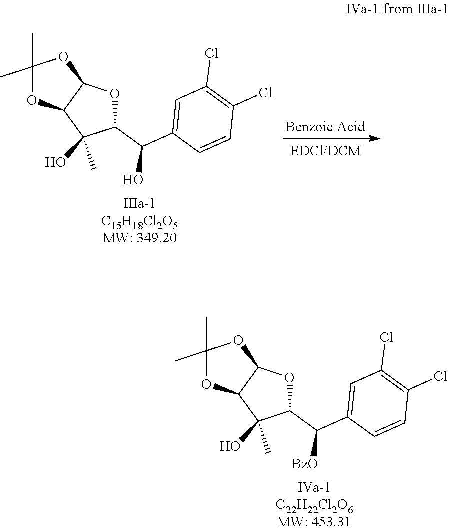

Benzoic acid (259.5 g, 2.12 mol, 1.10 eq) and anhydrous dichloromethane (10.1 L) were combined in a dry 20 L 4-neck glass reactor under N₂ and stirred to give a clear solution. EDCI.HCl (739.5 g, 3.85 mol, 2.00 eq) and DMAP (471.0 g, 3.85 mol, 2.00 eq) were slowly added and stirred until solution was clear again. The temperature was kept between 15-30° C. IIIa-1 (672.8 g, 1.93 mol, 1.00 eq) was then added to the solution and stirred for about 10 hours at 15-30° C. PCT was not ≤1.0 A % of IIIa-1 vs. IVa-1 by HPLC, so additional benzoic acid (12.01 g, 0.098 mol, 0.05 eq) was added and stirred. After 30 minutes, the reaction was complete and a HCl solution (1.0 M in water, 8.8 L) was slowly added to the mixture while maintaining a temperature at 15.0-30° C. and stirred for about 1 h. The organic phase was separated, and the acidic aqueous layer was discarded if no product was detected. The organic phase was then charged with NaHCO₃ solution (5% in water, 6.7 L) in the reactor and stirred. The organic phase was separated, to which Na₂SO₄ (1009.5 g) was then added. The batch was filtered through a 200 mm Buchner funnel and the cake was washed with 1.0 L of DCM. The cake was discarded. The filtrate and wash were combined and then condensed in a 20 L rotavapor at about 50° C. under vacuum to yield IVa-1 (898.0 g, 99.8%) with 97.4% purity via HPLC (Area %). ¹H NMR (400 MHz, CD₃OD-d4): δ 8.13 (m, 2H), 7.60 (m, 2H), 7.46 (m, 3H), 7.39 (d, J=12.0 Hz, 1H), 5.87 (d, J=12.0 Hz, 1H), 5.69 (d, J=4.0 Hz, 1H), 4.30 (d, J=8.0 Hz, 1H), 4.21 (d, J=4.0 Hz, 1H), 1.52 (s, 3H), 1.32 (s, 3H), 1.25 (s, 3H).

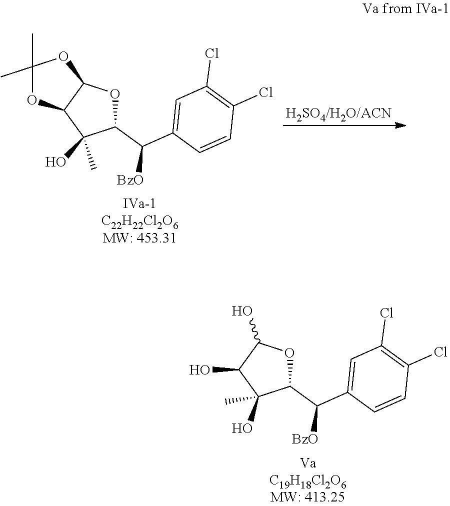

A mixture of IVa-1 (876.0 g, 1.93 mol, 1.0 eq) in CH₃CN/H₂O/H₂SO₄=16/8/0.5 (8.8 L) was stirred at 45-55° C. for 4 h. The reaction mixture was cooled to 0-5° C., and the pH was adjusted to 5-6 with 10% aq. NaOH (2.3 L), and then adjusted to between 7-8 with 10% aq. NaHCO₃ (1.4 L), with the reaction temperature being maintained <30° C. The solution was concentrated by distillation at 40-50° C. under vacuum to remove most of the CH₃CN. The mixture was extracted with ethyl acetate (2×5.4 L). The combined organic layers were washed with 10% aq. NaCl (4.4 L) and dried over anhydrous Na₂SO₄ (1315.0 g). The solid was removed by filtration and the cake was washed with EA (1.3 L). The filtrate was concentrated at 40-50° C. under vacuum to afford the product Va (827.5 g, Yield: >100%).

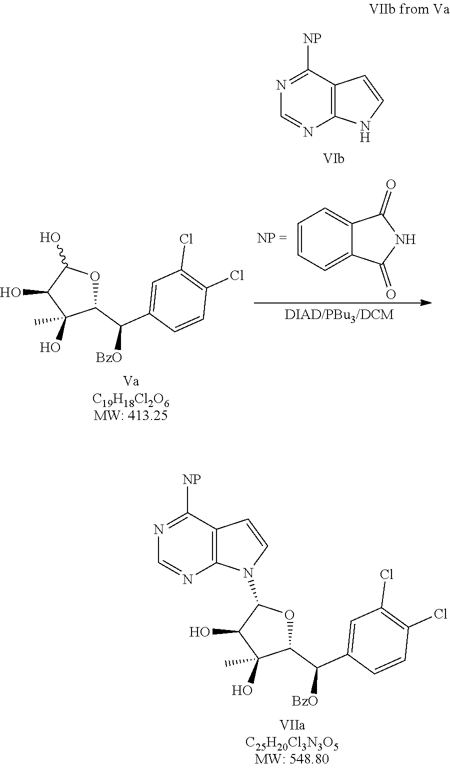

To a clean and dry glass reactor was added VIb (442.0 g, 1.00 eq) and DCM (19.9 L) under a nitrogen atmosphere. Tri(n-butyl)phosphine (846.1 g, 2.50 eq) and diisopropyl azodicarboxylate (845.6 g, 2.50 eq) were added successively at 1.2° C. The mixture was stirred at 5-10° C. for 0.75 h. After 0.75 h, a solution of Va (794.9 g, 1.15 eq) in THF (4.0 L) was added slowly to the above mixture while keeping the temperature below 15° C. The reaction mixture was stirred at 10-20° C. for 2 h. The mixture was quenched with 20% aq. NH₄Cl solution (6.4 L) and the mixture was separated. The aqueous layer was extracted with DCM (4.0 L). The combined organic layers were washed with 10% aq. NaCl solution (6.4 L) and dried over anhydrous Na₂SO₄ (1192 g). The mixture was filtered, washed with DCM (1.2 L), and concentrated under reduced pressure to afford crude VIIb (3146.5 g, 275%).

LC-MS calc. for C₃₃H₂₄Cl₂N₄O₇ CHO₂ [M+HCOO]⁻: m/z=703.1; Found:703.4.

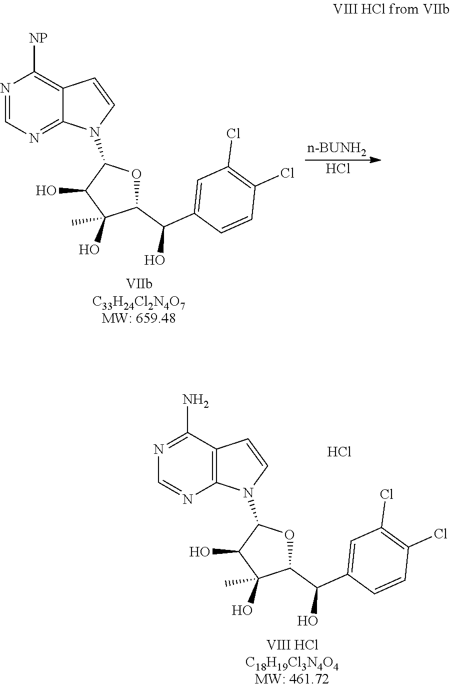

To a clean and dry 4-neck glass reactor with mechanical stirring, condenser and thermocouple were added the crude VIIb (2934 g) and MeOH (5.3 L). Next n-butyl amine (710 g) was added to the reaction. The mixture was warmed to 65-75° C. and stirred at that temperature for ≥48 h. The reaction mixture was transferred to a rotavapor and distilled at 40-50° C. under vacuum until no solvent can be distilled. The batch was transferred with EtOH (6.4 L) to a reactor and stirred to afford a clear solution. To the solution was added con. HCl (0.47 L) slowly to maintain temperature <30° C. The reaction was stirred at 15-25° C. for ≥3 h. The batch was filtered through a funnel and the filter cake was washed with EtOH (2.1 L). The filter cake was dried on the funnel by pulling air through for 15 min. The filter cake was transferred to a reactor and EtOH (3.2 L) was added to the reactor. The mixture was stirred at 15-25° C. for ≥2 h. The batch was filtered again through a funnel and the filter cake was washed with EtOH (2.1 L). The filter cake was dried on the funnel by pulling air through for 30 min. The batch was further dried in a vacuum oven at <50° C. under vacuum until the weight loss was <2.0% over a period of ≥3 h. The product obtained was VIII HCl. ¹H NMR (500 MHz, DMSO-d6): δ8.35 (s, 1H), 7.85 (d, J=3.7 Hz, 1H), 7.58 (d, J=2.0 Hz, 1H), 7.49 (d, J=8.3 Hz, 1H), 7.35 (dd, J=2.0, 8.4 Hz, 1H), 7.01 (d, J=3.7 Hz, 1H), 6.00 (d, J=8.2 Hz, 1H), 5.92 (s, 1H), 4.78 (d, J=7.9 Hz, 1H), 4.33 (d, J=8.2 Hz, 1H), 3.93 (d, J=7.9 Hz, 1H), 2.06 (s, 1H), 1.29 (s, 3H).

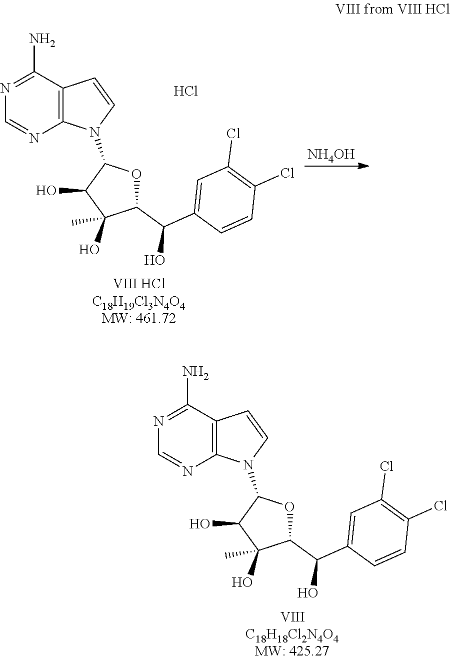 [SMILES: C[C@@]1(O)[C@@H]([C@H](O)c2ccc(Cl)c(Cl)c2)O[C@@H](n2ccc3c(N)ncnc32)[C@@H]1O ; Cl ; "NH4OH" ; C[C@@]1(O)[C@@H]([C@H](O)c2ccc(Cl)c(Cl)c2)O[C@@H](n2ccc3c(N)ncnc32)[C@@H]1O]

To a clean and dry 4-neck glass reactor with mechanical stirring, condenser and thermocouple was charged VIII HCl (453.0 g) and water (3.6 L). The mixture was stirred at 15-35° C. for 1 h. Aqueous ammonium hydroxide solution (0.11 L) was added while maintaining the reaction temperature at 20° C. until the batch reached pH 7-9. The mixture was stirred at 15-35° C. for 1 h. The batch was filtered, and the filter cake was washed with water (0.9 L). The cake was dried on the funnel by pulling air through the cake until no solvent dripping was observed, then transferred to blast drying oven and dried at 60° C. until the weight loss is 1.0% between weight checks taken 3 hours apart. The dried cake was the VIII free base. ¹H NMR (400 MHz, DMSO-d6): δ8.04 (s, 1H), 7.61 (d, J=1.75 Hz, 1H), 7.51 (d, J=8.77 Hz, 1H), 7.42 (d, J=3.51 Hz, 1H), 7.38 (dd, J=1.75, 8.33 Hz, 1H), 7.07 (br s, 2H), 6.55-6.64 (m, 2H), 5.85 (d, J=8.33 Hz, 1H), 5.27 (d, J=7.45 Hz, 1H), 4.78-4.86 (m, 2H), 4.43 (t, J=7.89 Hz, 1H), 4.01 (d, J=6.14 Hz, 1H), 1.18 (s, 3H).

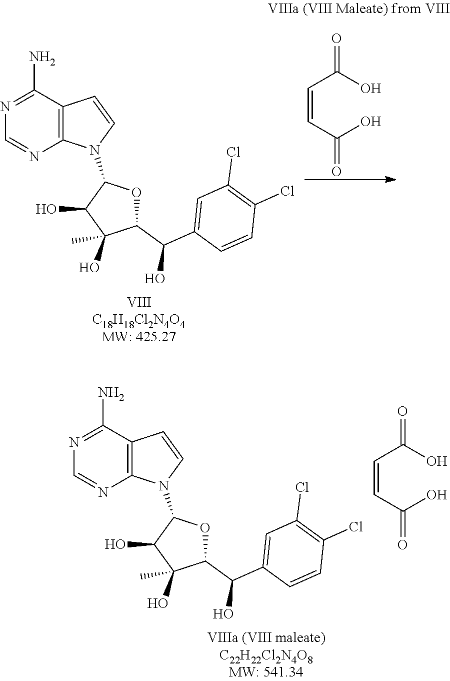

To a clean and dry reactor under nitrogen flow was charged VIII (4200 g) and methanol (32.7 L). The batch was heated to 20-45° C. and stirred to form a clear solution. The batch was filtered through a filter loaded with celite (4087 g) and washed with methanol (16.3 L). The filtrate and wash were transferred to a rotary evaporator through an in-line filter and distilled at ≥60° C. under vacuum until the distillation stopped. Filtered ethanol (6.2 L) was charged to the rotary evaporator and distilled at ≥60° C. under vacuum until the distillation stopped. The solid (VIII free base) was mixed with filtered ethanol (36.8 L) and transferred to a reactor. The batch was heated to 35-50° C. A polished filtered solution of maleic acid (1226 g) in ethanol (12.3 L) was added to the reaction and the batch temperature was maintained at 35-50° C. The batch was stirred at 35-50° C. for ≥30 minutes, cooled to 15-30° C., then stirred at 15-30° C. for ≥3 hours. The solid was filtered and the filter cake was washed with filtered ethanol (12.3 L). The product was dried by pulling air through the filter cake until no dripping was observed. Next product was transferred to drying trays and further dried under ambient air conditions. The product was further dried under vacuum at ≥45° C. until it reached a constant weight. The product was ground with a spatula and passed through a 60-mesh sieve.

The resulting solid was VIIIa (VIII Maleate). ¹H NMR (500 MHz, DMSO-d6): δ8.19 (s, 1H), 7.81 (s, 1H), 7.61 (dd, J=2.8, 17.5 Hz, 2H), 7.50 (d, J=8.3 Hz, 1H), 7.36 (dd, J=2.0, 8.4 Hz, 1H), 6.76 (d, J=3.5 Hz, 1H), 6.35-6.19 (m, 1H), 6.14 (s, 2H), 5.92 (d, J=8.2 Hz, 1H), 5.40-5.23 (m, 1H), 4.88 (s, 1H), 4.79 (d, J=7.2 Hz, 1H), 4.37 (d, J=8.2 Hz, 1H), 3.97 (d, J=7.2 Hz, 1H), 1.23 (s, 3H).

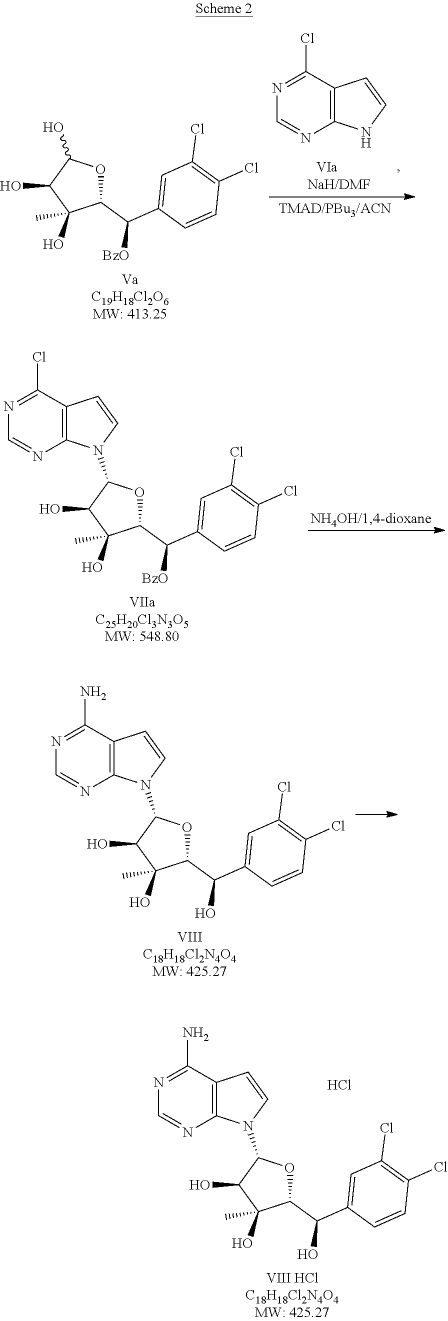

### Synthesis of (R)-((2R,3S,4R,5R)-5-(4-chloro-7H-pyrrolo[2,3-d]pyrimidin-7-yl)-3,4-dihydroxy-3-methyltetrahydrofuran-2-yl)(3,4-dichlorophenyl)methyl benzoate (VIIa)

To an oven dried 2 L 3-necked round bottom flask was charged compound Va (37.2 g, 90.02 mmol, 1.0 eq) and acetonitrile (929 ml) under N₂. P(n-Bu)₃ (38.23 mL, 153.03 mmol, 1.7 eq) was added, followed by TMAD (23.2 g, 135.03 mmol, 1.5 eq) at RT. The resulting mixture was stirred for 1 hr at RT. The solid was then removed by vacuum filtration under the protection of nitrogen. The filtrate was transferred back to flask.

In parallel, a 250 mL 3-necked round bottom flask was charged with anhydrous DMF (74 mL). NaH (60% in mineral oil, 5.04 g, 126.03 mmol, 1.4 eq) was added, followed by the addition of 4-chloro-7H-pyrrolo[2,3-d]pyrimidine (20.736 g, 135.03 mmol, 1.5 eq) portion wise at RT. The mixture was also stirred at rt for 1 hr. It was then transferred by cannular to the 2 L flask. The resulting mixture was stirred at RT overnight. An aliquot was analyzed by HPLC. Upon completion, the reaction was carefully quenched by adding 1M HCl to adjust the pH to 7. The reaction mixture was concentrated under vacuum to remove acetonitrile. The residue was extracted with MTBE (3×600 mL). The organic layer was washed with 0.5 N HCl (5×1000 mL), saturated NaHCO₃ (200 mL), and brine (200 mL). It was dried over anhydrous Na₂SO₄, filtered and concentrated under reduced pressure to provide crude VIIa, which was directly used for the next step without further purification. calc. for C₂₅H₂₁Cl₃N₃O₅ [M+H]⁺: m/z=548.05; Found:547.70.

### Synthesis of (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol (VIII)

Crude compound VIIa (ca. 86.02 mmol) was dissolved in aqueous NH₄OH solution (28-30%, 737 mL) and 1,4-dioxane (420 mL). The resulting mixture was stirred at 100° C. in a Parr reactor for 36 hrs. HPLC analysis indicated all starting material was consumed. The reaction mixture was concentrated under reduced pressure. The residue was diluted with H₂O (500 mL) and ethyl acetate (1000 mL). The layers were separated. The organic layer was washed with H₂O (2×500 mL). It was dried over anhydrous Na₂SO₄ and concentrated under reduced pressure. The residue was slurried with EtOAc/heptane (1/9, 1000 mL) for 24 hrs. The solid was collected by vacuum filtration. The slurry process was repeated once more time with same amount of solvent. The solid was dried under vacuum at 40° C. to afford crude VIII as a light brown solid (24.95 g). Calc. for C₁₈H₁₉Cl₂N₄O₄ [M+H]⁺: m/z=425.08; Found:424.7.

VIII HCl salt formation: Crude VIII (24.95 g) was dissolved in MeOH (274 mL), and 33 mL of HCl/IPA solution (5M-6M) was added to the solution dropwise. The mixture was stirred at RT for 4 hrs. The slurry was filtered under reduced pressure. The filter cake was washed with IPA (2×15 mL) to provide 11.73 g of VIII HCl salt (purity >98%). LC-MS calc. for C₁₈H₁₉Cl₂N₄O₄ [M+H]⁺: m/z=425.08; Found:424.7.

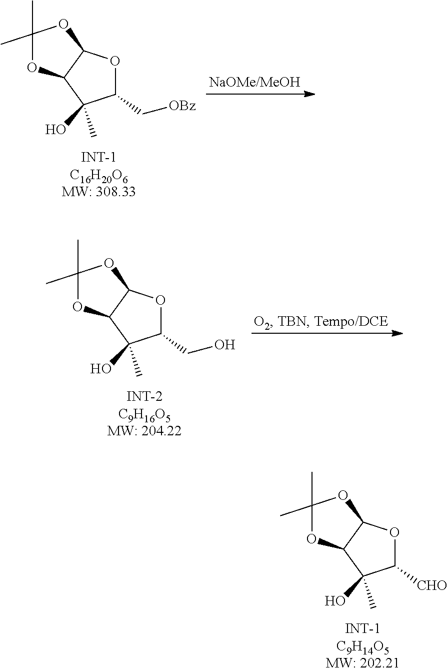

### Synthesis of (3aR,5R,6R,6aR)-5-(hydroxymethyl)-2,2,6-trimethyltetrahydrofuro[2,3-d][1,3]dioxol-6-ol (INT-2)

To a solution of compound INT-1 (1.69 Kg, 5.48 mol) in MeOH (8.5 L) was added NaOMe (14.8 g, 274 mmol, 0.05 eq.) at 25° C. The reaction was stirred at room temperature for 16 hrs. Aliquot analysis by HPLC indicated that the starting material is less than 1.5%, and no further improvement was observed after additional 4 hrs. The reaction mixture was concentrated under reduced pressure at 45° C., and then diluted with pre-heated solvent mixture (20 L, 10% ethyl acetate in petroleum ether). The mixture was allowed to slowly cool to room temperature with slow stirring. It was filtered and washed with 10% ethyl acetate/petroleum ether (˜2 L) to afford compound INT-2 as a light brown solid (1017 g, yield 91%.), which was directly used for the next step without further purification. ¹H NMR (400 MHz, CDCl₃-d3): δ 5.78 (d, J=3.8 Hz, 1H), 4.12 (d, J=3.8 Hz, 1H), 3.88 (d, J=3.6 Hz, 1H), 3.81 (m, 2H), 1.58 (s, 3H), 1.36 (s, 3H), 1.17 (s, 3H).

### Synthesis of (3aR,5S,6R,6aR)-6-hydroxy-2,2,6-trimethyltetrahydrofuro[2,3-d][1,3]dioxole-5-carbaldehyde (Ia-1)

To a Teflon-lines 316 L stainless steel autoclave (1 L) with mechanical stirring, were added 1,2-dichloroethane (DCE, 600 mL), compound INT-2 (102.1 g, 0.5 mol), TEMPO (1.56 g, 10 mmol, 0.02 eq.) and TBN (3.09 g, 30 mmol, 0.06 eq.). Then the autoclave was closed and charged with oxygen to 0.2 MPa. The autoclave was placed in an oil bath, which was preheated to 80° C. Oxygen was recharged to maintain the pressure. After 9 hrs, the barometer was constant which indicated that the reaction was finished. The autoclave was taken out from the oil bath, cooled to room temperature and carefully depressurized. GC analysis indicated that all starting material was consumed, and there is about 6% over-oxidized byproduct (the acid). The crude was passed through a short silica plug to remove the acid byproduct, and the filtrate was concentrated to afford compound Ia-1 as a light yellow solid (70.7 g, yield 69.9%.). ¹H NMR (500 MHz, DMSO-d6): δ 9.56 (d, J=0.9 Hz, 1H), 5.80 (d, J=3.4 Hz, 1H), 5.59 (s, 1H), 4.32 (s, 1H), 4.17 (d, J=3.4 Hz, 1H), 1.46 (s, 3H), 1.27 (s, 3H), 1.03 (s, 3H).

### HPLC Methods:

A. HPLC Method for the Separation of the Enantiomer-VIII (Ent-VIII) from VIII and the Quantitation of Ent-VIII in VIII Maleate Drug Substance Using a Chiral HPLC Method.

An HPLC system equipped with quaternary or binary pump, autosampler, thermostated column compartment, and UV/Vis detector is employed in the analysis. The HPLC column is a Chiralpak IA, 5 μm, 4.6×250 mm (Part No. 80325).

Mobile Phase: Hexanes: EtOH: MeOH (80:10:10). This mixture may be prepared by mixing 800 mL of Hexanes, 100 mL of EtOH and 100 mL of MeOH in 1 L bottle, mixing well and degassing.

HPLC Conditions:
Flow Rate (mL/min.):1.0
Injection volume (μL): 10
UV Detector Wavelength (nm): 230 nm
Total Run Time (minutes): 22
Column Temperature (° C.): 30

### B. HPLC Method for the Identification and Determination of VIII and Related Substances (Including Starting Materials, Intermediates and Impurities) in VIII Maleate Drug Substance by Reverse Phase HPLC.

An HPLC system equipped with quaternary or binary pump, autosampler, thermostated column compartment, and UV/Vis detector is employed in the analysis. The HPLC column is a Zorbax XDB-C18, 3.5 μm, 4.6×150 mm. Part number: 963967-902.

Mobile Phases
Mobile Phase A: 0.05% TFA (% v/v) in DI water. Example preparation: add 0.5 mL of TFA to 1.0 L of DI water. Degas as needed.
Mobile Phase B: 0.05% TFA (% v/v) in acetonitrile. Example preparation: add 0.5 mL of TFA to 1.0 L of acetonitrile. Degas as needed.

HPLC Conditions:

| Flow Rate | 1.0 | | |
|---|---|---|---|
| (mL/min.) | | | |
| Injection volume | 5 | | |
| (μL): | | | |
| UV Detector | 226 nm | | |
| Wavelength | | | |
| (nm): | | | |
| Data Acquisition | 20 | | |
| Time (minutes): | | | |
| Total Run Time | 25 | | |
| (minutes): | | | |
| Column | 40 | | |
| Temperature | | | |
| (° C.): | | | |
| | Time (min.) | % MPA | % MPB |
| Pump Gradient: | 0 | 95 | 5 |
| | 16 | 5 | 95 |
| | 20 | 5 | 95 |
| | 20.1 | 95 | 5 |
| | 25 | 95 | 5 |

1. A process for preparing a compound of formula (III), comprising reacting a compound of formula (I) with a compound of formula (II) in the presence of an organic solvent: 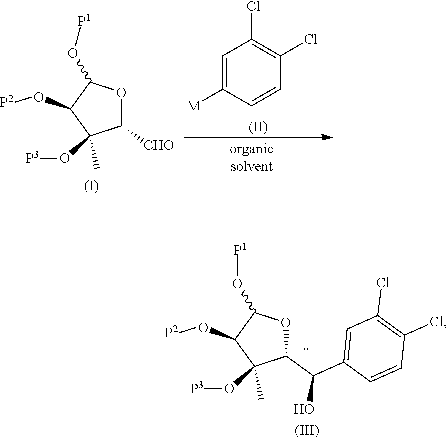
wherein
M is a metal atom-containing moiety, a boronate ester, or a boronic acid;
P¹ and P² are each, independently, a hydroxyl protecting group;
or P¹ and P² together with the oxygen atoms to which they are attached form a 1,2-dihydroxyl protecting group; and
P³ is H or a hydroxyl protecting group;
or P² and P³ together with the oxygen atoms to which they are attached form a 1,2-dihydroxyl protecting group.

2. The process of claim 1, wherein the compound of formula (I) is the compound of formula (Ia) and the compound of formula (III) is the compound of formula (IIIa)

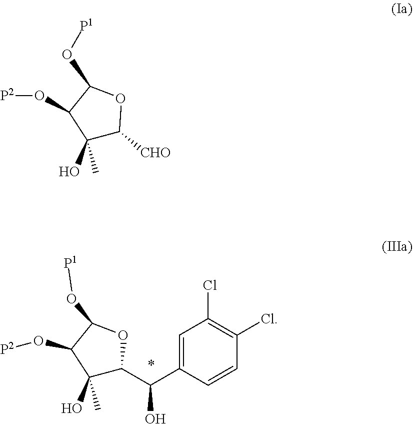

3. The process of claim 1, wherein P¹ and P² together with the atoms to which they are attached, form a 1,2-dihydroxyl protecting group.

4. (canceled)

5. The process of claim 3, wherein the compound of formula (Ia) is formula (Ia-1):

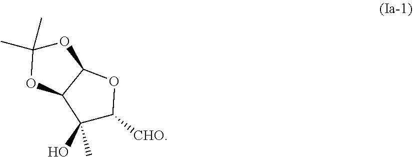

6. The process of claim 1, wherein the compound of formula (III) is the compound of formula (IIIa-1):

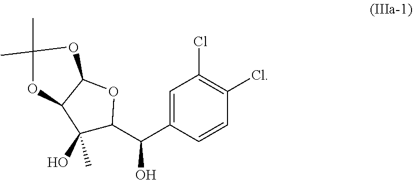 [SMILES: CC1(C)O[C@H]2OC([C@H](O)c3ccc(Cl)c(Cl)c3)[C@@](C)(O)[C@H]2O1]

7. The process of claim 1, wherein the compound of formula (I) is the compound of formula (Ib) and the compound of formula (III) is the compound of formula (IIIb)

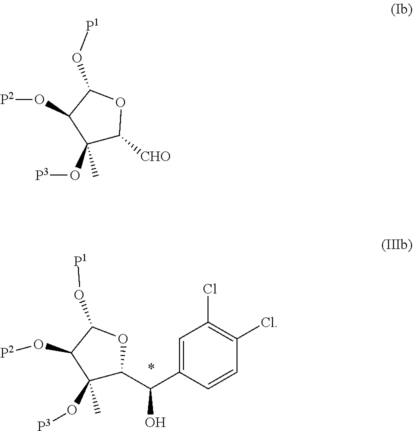

8. The process of claim 1, wherein P² and P³ together with the atoms to which they are attached, form a 1,2-dihydroxyl protecting group.

9. (canceled)

10. The process of claim 8, wherein the compound of formula (Ib) is the compound of formula (Ib-1):

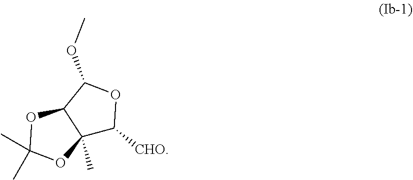

11. The process of claim 7, wherein the compound of formula (IIIb) is the compound (IIIb-1):

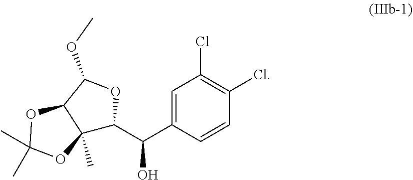 [SMILES: CO[C@@H]1O[C@H]([C@H](O)c2ccc(Cl)c(Cl)c2)[C@@]2(C)OC(C)(C)O[C@@H]12]

12. The process of claim 1, wherein reacting a compound of formula (I) with a compound of formula (II) in the presence of an organic solvent is carried out in the presence of a Lewis acid.

13. (canceled)

14. The process of claim 12, wherein the Lewis acid is ZnCl₂.

15. The process of claim 1, wherein M is Li, MgL, ZnL, NiL₃, BL₂, CuL, SnL₃, Pd(L)₂, or Pd(L)₄, wherein L is a ligand.

16. (canceled)

17. The process of claim 1, wherein the compound of formula (II) is the compound of formula (IIa):

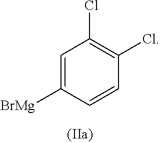

18. The process of claim 1, wherein the organic solvent is diethyl ether, t-butyl methyl ether, or tetrahydrofuran, or a combination thereof.

19. The process of claim 1, wherein the enantiomeric excess at the benzylic carbon atom (*) in the compound of formula (III) is at least 80%, or at least 90%, or at least 95%, or at least 98%, or at least 99%, or at least 99.5%, or at least 99.8%, or at least 99.9%.

20-26. (canceled)

27. The process according to claim 1, wherein the compound of formula (III) is formed in at least 80% diastereomeric excess, or at least 90% diastereomeric excess, or at least 95% diastereomeric excess, or at least 98% diastereomeric excess, or at least 99% diastereomeric excess, or at least 99.5% diastereomeric excess, or at least 99.8% diastereomeric excess, or at least 99.9% diastereomeric excess.

28-34. (canceled)

35. The process of claim 1, further comprising treating the compound of formula (III) with a P⁴-Reagent System for a time and under conditions sufficient to provide a compound of formula (IV): 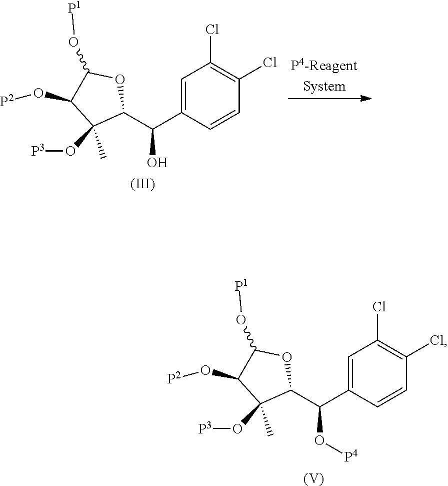
wherein the P⁴-Reagent System is a reagent that reacts with the compound of formula (III) to produce the compound of formula (IV), wherein P⁴ is an acid-stable, base-labile hydroxyl protecting group, and when P³ in formula (III) is H, P³ in formula (IV) is H or, together with P⁴, is an acid-stable, base-labile 1,3-dihydroxyl protecting group.

36. The process of claim 35, wherein P⁴ is 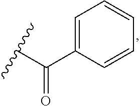 [SMILES: *C(=O)c1ccccc1]
and wherein the P⁴-Reagent System is benzoic acid, an additive, and a coupling agent in the presence of an organic solvent.

37. The process of claim 35, wherein the compound of formula (III) is a compound of formula (IIIa) and the compound of formula (IV) is a compound of formula (IVa):

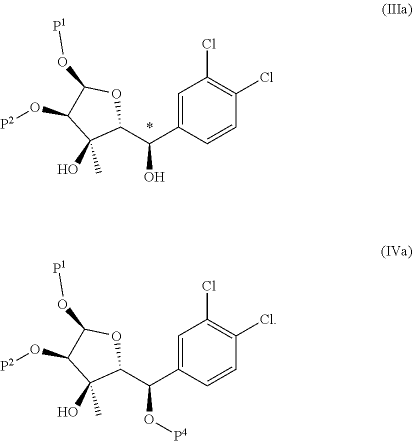

38. The process of claim 37, wherein the compound of formula (IVa) is the compound of formula (IVa-1):

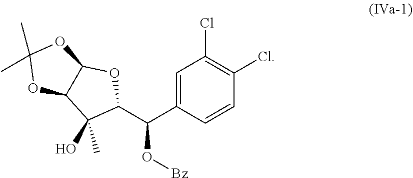

39. The process of claim 35, wherein the compound of formula (III) is a compound of formula (IIIb) and the compound of formula (IV) is a compound of formula (IVb)

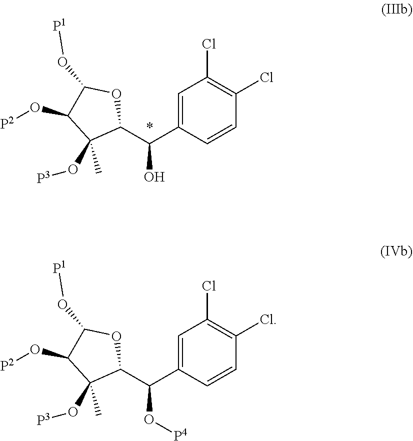

40. The process of claim 39, wherein the compound of formula (IVb) is the compound of formula (IVb-1):

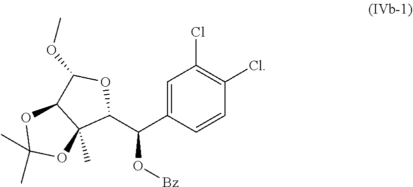

41. The process of claim 35, further comprising treating the compound of formula (IV) with aqueous acid to provide a compound of formula (V): 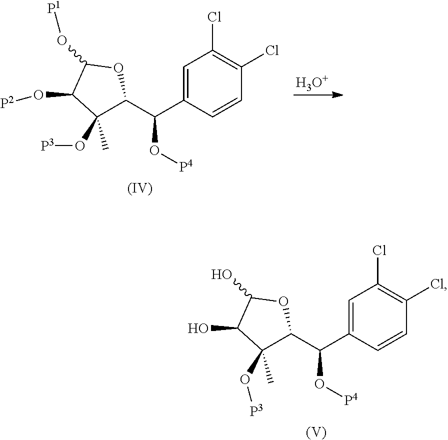
wherein P³ in the compound of formula (V) is H, or together with P⁴, forms an acid-stable, base-labile 1,3-dihydroxyl protecting group.

42. (canceled)

43. The process of claim 41, wherein the compound of formula (V) is the compound (Va):

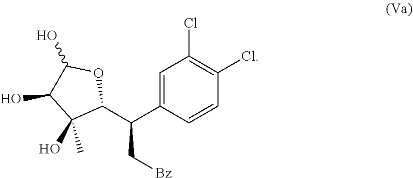

44. The process of claim 41, further comprising reacting the compound of formula (V) with a compound of formula (VI), or a basic salt thereof, in the presence of a phosphine ((R²)₃P), an azodicarboxylate or derivative thereof, in the presence of an organic solvent, to produce a compound of formula (VII): 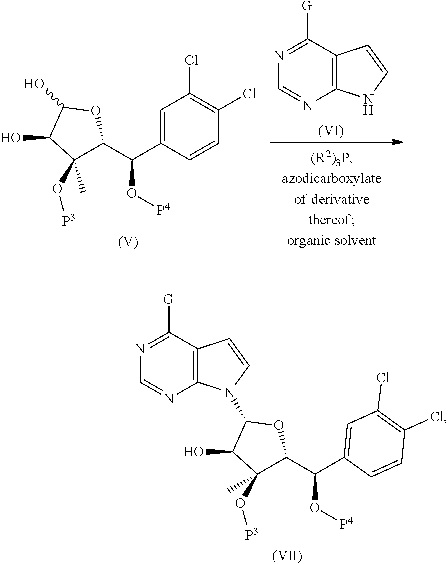
wherein G is a halogen or a masked amino group, each R² is independently C₁-C₆alkyl or aryl, and P³ is H, or together with P⁴, is an acid-stable, base-labile 1,3-dihydroxyl protecting group.

45. The process of claim 44, wherein the phosphine is triphenylphoshine or tributylphosphine, and the azodicarboxylate or derivative thereof is diethylazodicarboxylate (DEAD), diisopropylazodicarboxylate (DIAD), or tetramethyl azodicarboxamide (TMAD).

46. The process of claim 44, wherein G is a halogen.

47. The process of claim 46, wherein the compound of formula (VI) is the compound of formula (VIa) or a basic salt thereof:

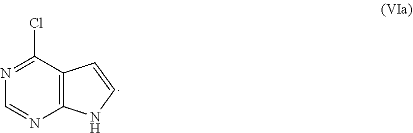

48. The process of claim 44, wherein the compound of formula (VII) is the compound of formula (VIIa):

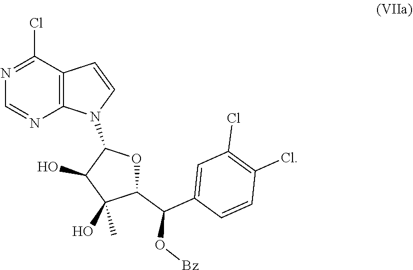

49. (canceled)

50. The process according to claim 44, wherein G is isoindol-2-yl-1,3-dionyl.

51. The process according to claim 50, wherein the compound of formula (VI) is the compound of formula (VIb), or a basic salt thereof:

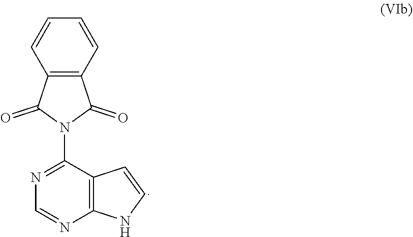 [SMILES: O=C1c2ccccc2C(=O)N1c1ncnc2[nH]ccc12]

52. The process of claim 49, wherein the compound of formula (VII) is the compound of formula (VIIb):

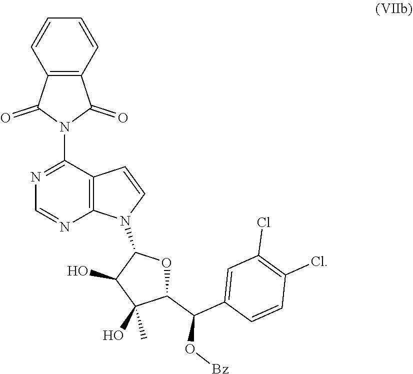

53. The process of claim 41, further comprising converting the compound of formula (V) to an epoxide of formula (IX) by reacting the compound of formula (V) with a phosphine ((R²)₃P), an azodicarboxylate or derivative thereof, in the presence of an organic solvent: 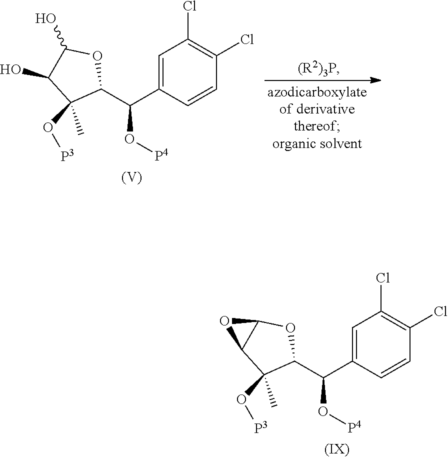
wherein each R² is independently C₁-C₆alkyl or aryl, and P³ is H, or wherein P³ and P⁴ together form an acid-stable, base-labile 1,3-dihydroxyl protecting group.

54. The process of claim 53, further comprising reacting the compound of formula (IX) with a compound of formula (VI), or a basic salt thereof, in an organic solvent to give a compound of formula (VII): 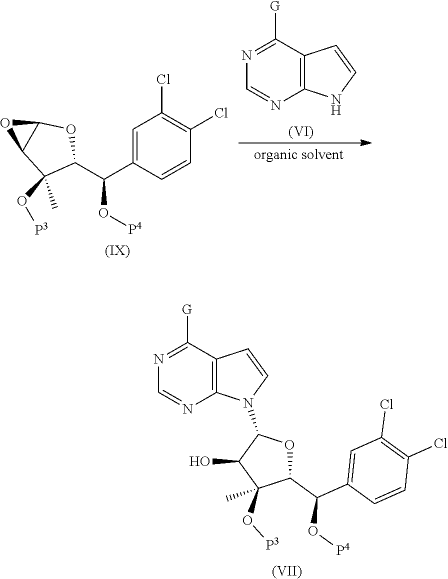
wherein G is a halogen or a masked amino group, and P³ is H, or wherein P³ and P⁴ together form an acid-stable, base-labile 1,3-dihydroxyl protecting group.

55. The process of claim 48, further comprising converting the compound of formula (VII), wherein G is halogen and P³ is H, or wherein P³ and P⁴ together form an acid-stable, base-labile 1,3-dihydroxyl protecting group, to a compound of formula (VIII): 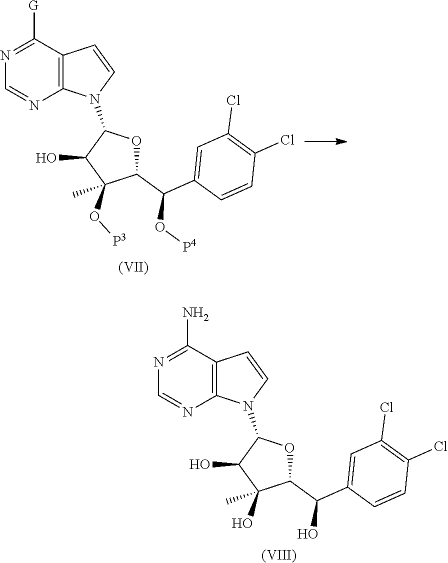
by reacting the compound of formula (VII) with ammonia.

56. The process of claim 49, further comprising converting the compound of formula (VII), wherein G is a masked amino group and P³ is H, or wherein P³ and P⁴ together form an acid-stable, base-labile 1,3-dihydroxyl protecting group, to a compound of formula (VIII): 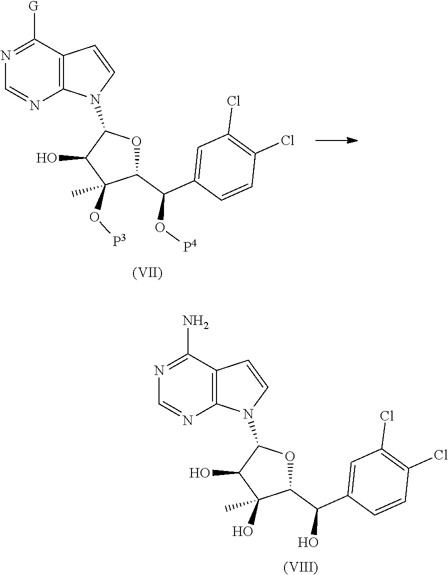
by reacting the compound of formula (VII) with a primary alkyl amine.

57. The process of claim 55, further comprising reacting the compound of formula (VIII) with HCl in a solvent to form an HCl salt of the compound of formula (VIII).

58. The process of claim 57, further comprising reacting the HCl salt of the compound of formula (VIII) with a base, preferably ammonium hydroxide, in a solvent, preferably water, to give the compound of formula (VIII) as a free base.

59. The process of claim 55, further comprising reacting the compound of formula (VIII) with an acid to form a pharmaceutically acceptable salt of the compound of formula (VIII).

60. The process of claim 59, wherein the pharmaceutically acceptable salt is the maleate salt.

61. The process of claim 12, wherein the compound of formula (III) has the formula (IIIa-1), the compound of formula (I) has the formula (Ia-1), the compound of formula (II) has the formula (IIa), and wherein the aprotic solvent is THE and the Lewis acid is ZnCl₂:

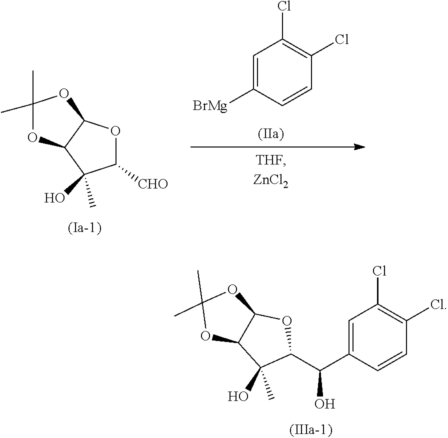

62. The process of claim 61, further comprising reacting the compound of formula (IIIa-1) with benzoic acid in the presence of 1-ethyl-3-(3-dimethylaminopropyl)carbodiimide (EDCI) or a salt thereof, DMAP, and an organic solvent to give the compound of formula (IVa-1):

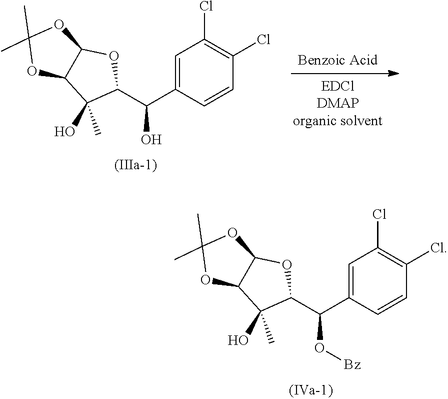

63. The process of claim 62, further comprising reacting the compound of formula (IVa-1) with an aqueous acid comprising a mineral acid to give the compound of formula (Va):

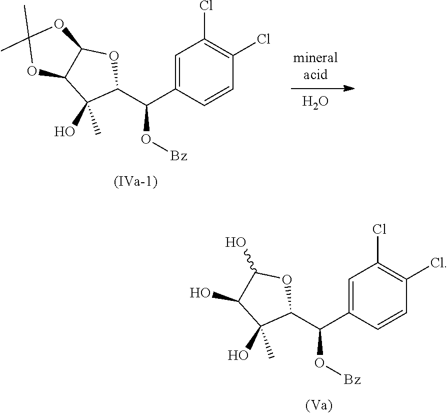

64. The process of claim 63, further comprising reacting the compound of formula (Va) with the compound of formula (VIa) or the sodium salt thereof, in the presence of tetramethylazodicarboxamide (TMAD) and tributylphosphine in organic solvent to give to the compound of formula (VIIa):

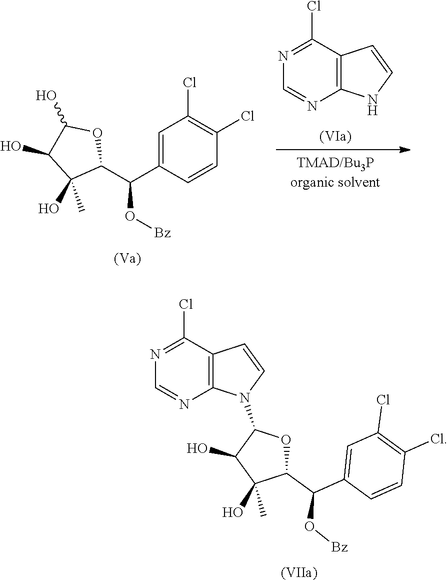

65. The process of claim 64, further comprising reacting the compound of formula (VIIa) with ammonia in an organic solvent to give the compound of formula (VIII):

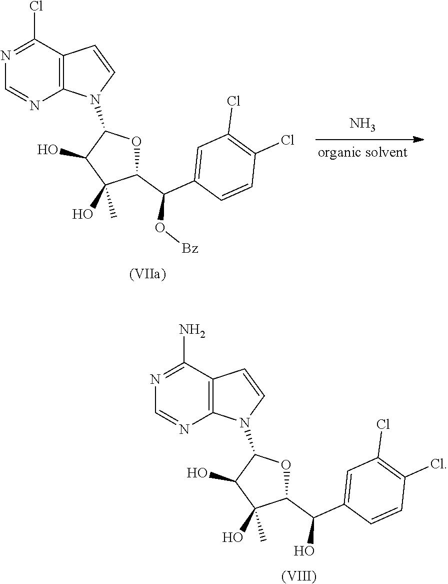

66. The process of claim 63, further comprising reacting the compound of formula (Va) with the compound of formula (VIb) in the presence of diisopropylazodicarboxylate (DIAD) and tributylphosphine in organic solvent to give the compound of formula (VIIb):

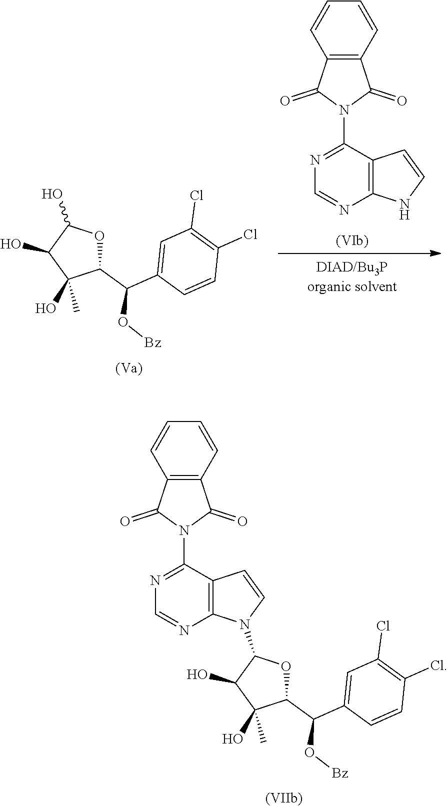

67. The process of claim 66, further comprising reacting the compound of formula (VIIb) with n-butyl amine in an organic solvent to give the compound of formula (VIII):

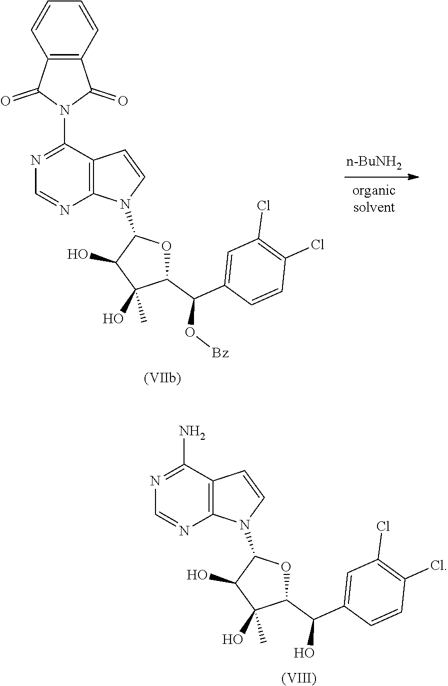

68. The process of either claim 65, further comprising reacting the compound of formula (VIII) with HCl in a solvent to produce the HCl salt of the compound of formula (VIII).

69. The process of claim 68, further comprising reacting the HCl salt of the compound of formula (VIII) with a base in a solvent to produce the compound of formula (VIII) as a free base.

70. The process of claim 65, further comprising reacting the compound of formula (VIII) with maleic acid in a solvent to produce a pharmaceutically acceptable salt of the compound of formula (VIII) that is a compound of formula (VIIIa):

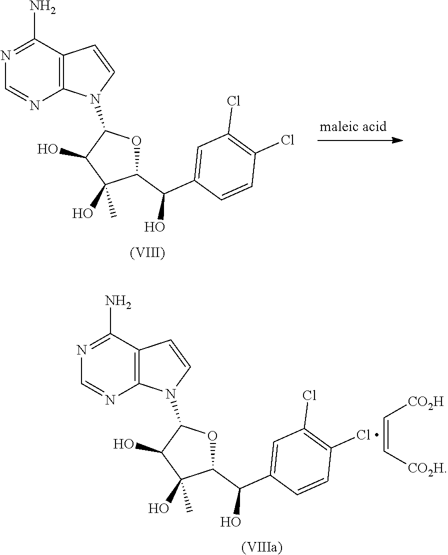

71. A process for preparing a compound of formula (VIII) 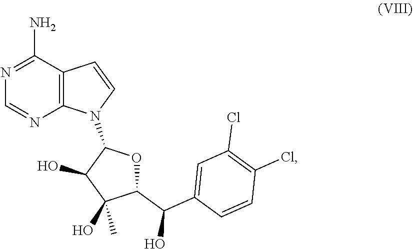 [SMILES: C[C@@]1(O)[C@@H]([C@H](O)c2ccc(Cl)c(Cl)c2)O[C@@H](n2ccc3c(N)ncnc32)[C@@H]1O]
or a pharmaceutically acceptable salt thereof, wherein said process comprises a process according to claim 1.

72. A process for preparing a compound of formula (VIII) comprising reacting a compound of formula (VII) with ammonia for a time and under conditions sufficient to produce the compound of formula (VIII): 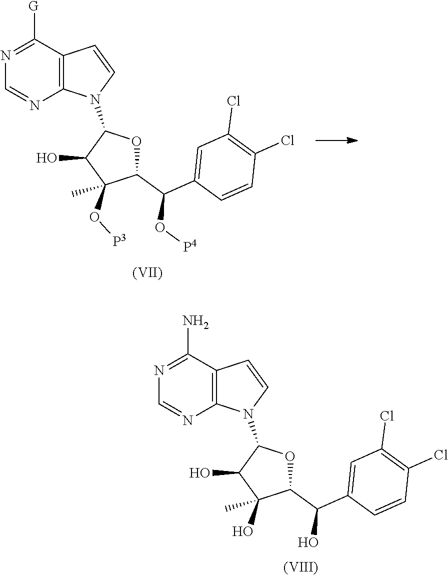
wherein
G is halogen;
P³ is H, and
P⁴ is an acid stable, base-labile hydroxyl protecting group, or P³ and P⁴ together form an acid-stable, base-labile 1,3-dihydroxyl protecting group.

73. A process for preparing a compound of formula (VIII) comprising reacting the compound of formula (VII) with a primary alkylamine for a time and under conditions sufficient to produce the compound of formula (VIII): 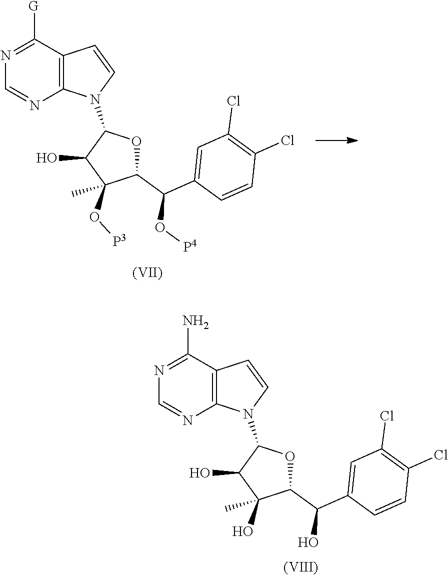
wherein
G is a masked amino compound;
P³ is H, and
P⁴ is an acid stable, base labile hydroxyl protecting group, or P³ and P⁴ together form an acid-stable, base-labile 1,3-dihydroxyl protecting group.

74. The process of claim 72 wherein the compound of formula (VII) is the compound of formula (VIIa):

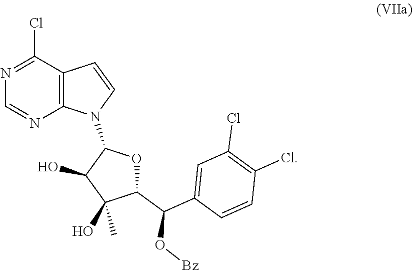

75. The process of claim 73 wherein the compound of formula (VII) is the compound of formula (VIIb):

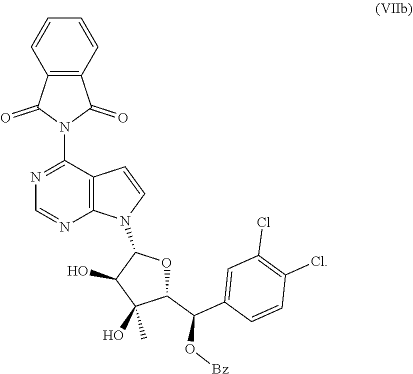

76. The process of claim 72, wherein the compound of formula (VII) is prepared by reacting the compound of formula (V) with a compound of formula (VI) or a basic salt thereof, in the presence of a phosphine, an azodicarboxylate or derivative thereof, and an organic solvent, for a time and under conditions sufficient to produce the compound of formula (VII): 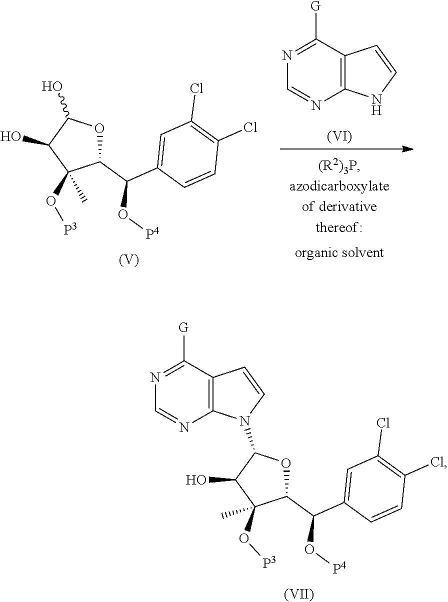
wherein each R² is independently C₁-C₆alkyl or aryl, and P³ is H, or P³ and P⁴ together form an acid-stable, base-labile 1,3-dihydroxyl protecting group.

77. The process of claim 76, wherein the compound of formula (V) is the compound of formula (Va):

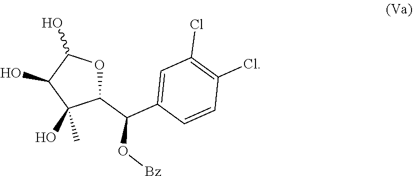

78. The process of claim 76, wherein the compound of formula (V) is prepared by treating the compound of formula (IV) with aqueous acid for a time and under conditions sufficient to produce the compound of formula (V): 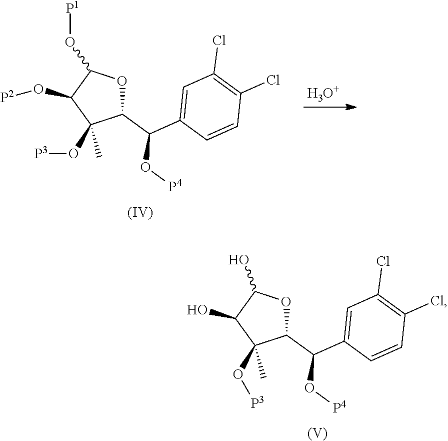
wherein P¹ and P² are each, independently, a hydroxyl protecting group; or P¹ and P² together with the oxygen atoms to which they are attached form a 1,2-dihydroxyl protecting group; and in formula (IV) P³ is H or a hydroxyl protecting group; or P² and P³ together with the oxygen atoms to which they are attached form a 1,2-dihydroxyl protecting group, and in Formula (V) P³ is H or P³ and P⁴ together form an acid-stable, base-labile 1,3-dihydroxyl protecting group.

79. The process of claim 78, wherein the compound of formula (IV) is the compound of formula (IVa-1):

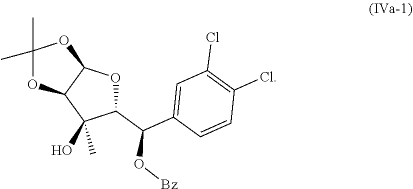

80. The process of claim 78, wherein the compound of formula (IV) is the compound of formula (IVb-1):

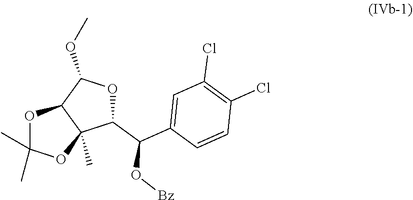

81. The process of claim 78, wherein the compound of formula (IV) is prepared by reacting a compound of formula (III) with a P⁴-Reagent System for a time and under conditions sufficient to provide a compound of formula (IV): 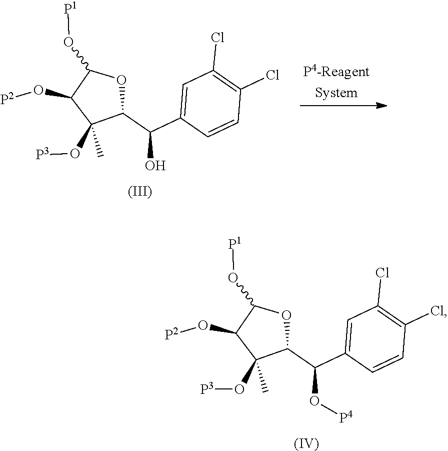
wherein the P⁴-Reagent System is a reagent that reacts with the compound of formula (III) to produce the compound of formula (IV), wherein P⁴ is a hydroxyl protecting group.

82. The process of claim 81, wherein the compound of formula (III) is a compound of formula (IIIa-1):

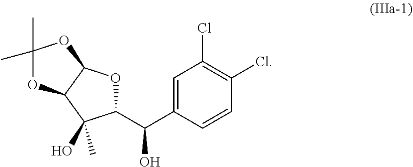 [SMILES: CC1(C)O[C@H]2O[C@H]([C@H](O)c3ccc(Cl)c(Cl)c3)[C@@](C)(O)[C@H]2O1]

83. The process of claim 81, wherein the compound of formula (III) is a compound of formula (IIIb-1):

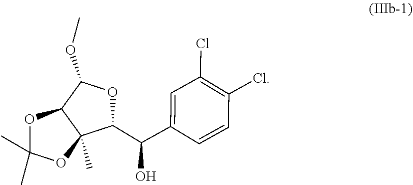 [SMILES: CO[C@@H]1O[C@H]([C@H](O)c2ccc(Cl)c(Cl)c2)[C@@]2(C)OC(C)(C)O[C@@H]12]

84. The process of claim 81, wherein the compound of formula (III) is prepared by reacting a compound of formula (I) with a compound of formula (II) in the presence of an organic solvent for a time and under conditions sufficient to produce the compound of formula (III): 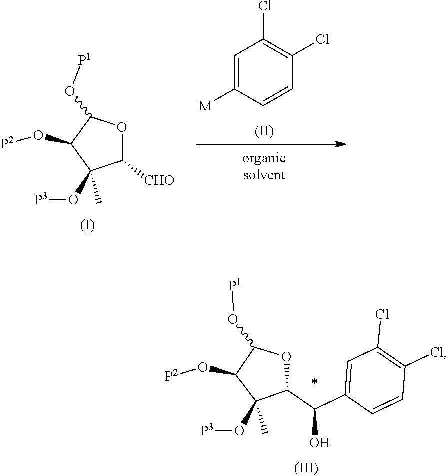
wherein M is a metal atom-containing moiety, a boronate ester, or a boronic acid, P¹ and P² are each, independently, a hydroxyl protecting group; or P¹ and P² together with the oxygen atoms to which they are attached form a 1,2-dihydroxyl protecting group; and P³ is H or a hydroxyl protecting group; or P² and P³ together with the oxygen atoms to which they are attached form a 1,2-dihydroxyl protecting group.

85. The process of claim 84, wherein the compound of formula (I) is a compound of formula (Ia-1):

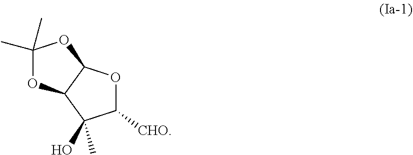

86. The process of claim 84, wherein the compound of formula (I) is the compound of formula (Ib-1):

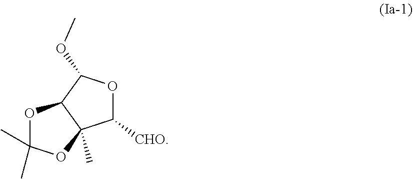

87. The process of claim 84, wherein the compound of formula (II) is the compound of formula (IIa):

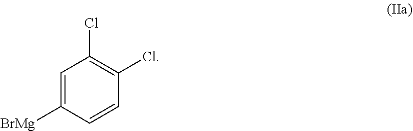

88. The process of claim 84, wherein the conditions sufficient to produce the compound of formula (III) comprise conducting the reaction in the presence of a Lewis acid.

89. The process of claim 88, wherein the Lewis acid is ZnCl₂.
